(19) United States
(12) Patent Application Publication
(10) Pub. No.: US 2026/0056204 A1
(43) Pub. Date: Feb. 26, 2026

(54) COMPOSITIONS AND METHODS FOR CHEMOPROTEOMIC REAGENT SYNTHESIS AND APPLICATION

(71) Applicant: The Regents of the University of California, Oakland, CA (US)

(72) Inventors: Keriann Backus, Los Angeles, CA (US); Nikolas Burton, Los Angeles, CA (US)

(21) Appl. No.: 19/102,520

(22) PCT Filed: Aug. 18, 2023
(86) PCT No.: PCT/US2023/030581

§ 371 Date: Feb. 10, 2025

(51) Int. Cl. G01N 33/68; C07F 7/18; G01N 33/532

(52) U.S. Cl. CPC G01N 33/6848; C07F 7/1804; G01N 33/532; G01N 2458/15

(57) ABSTRACT

Disclosed herein are compositions and methods of synthesizing chemoproteomics capture reagents.

### RELATED APPLICATIONS

This application claims the benefit of priority to U.S. Provisional Patent Application No. 63/399,262, filed Aug. 19, 2022; the contents of which are hereby incorporated by reference in their entirety.

### STATEMENT OF GOVERNMENT SUPPORT

This invention was made with government support under Grant Number D19AP00041, awarded by the U.S. Department of Defense, Defense Advanced Research Projects Agency.

The government has certain rights in the invention.

### FIELD OF THE INVENTION

Embodiments of the present disclosure generally relate to reagents for and methods of conducting mass spectrometry-based chemoproteomics.

### BACKGROUND

Mass spectrometry-based chemoproteomics has emerged as a powerful technology for functional biology and drug discovery. Recent application of chemoproteomics screening methods have enabled the discovery of thousands of potentially druggable sites proteome-wide. A standard chemoproteomics workflow accomplishes these objectives by combining capture of labeled peptides using biotinylated enrichment handles with isotopic differentiation of sample treatment groups. Nearly all chemoproteomics capture reagents feature (1) a biotin or desthiobiotin moiety for capture on streptavidin, avidin, or neutravidin resin, and (2) a capture handle, which is typically either an azide or alkyne group to enable bioorthogonal conjugation by copper-catalyzed azide-alkyne cycloaddition (CuAAC) or ‘click’ chemistry or a reactive group such as iodoacetamide that directly labels reactive amino acid side chains (e.g. cysteine thiol). A highly useful addition to these reagents is the incorporation of a cleavable linker (e.g. enzymatic, chemical, or photochemical cleavable group) positioned between the biotin/desthiobiotin and the capture handle. Among all cleavable linkers, the dialkoxydiphenylsilane (DADPS) group has emerged as a favored reagent, which is now widely adopted in chemoproteomics. Despite its favorable properties for chemoproteomics, synthetic strategies for incorporation of DADPS moieties into enrichment reagents remain limited. Thus, there is an ongoing, unmet need for new synthetic strategies to synthesize chemoproteomics capture reagents.

### SUMMARY OF THE INVENTION

In one aspect, the present disclosure provides compounds of formula I or a salt thereof:

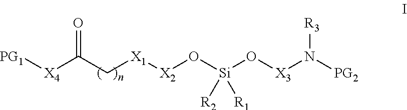

wherein,
X₁ and X₄ are each independently O, S, or NR₄;
X₂ and X₃ are each independently alkylene;
PG₁ is H, an oxygen protecting group, or a sequence of amino acids;
PG₂ is H, a nitrogen protecting group or a sequence of amino acids; and
R₁ and R₂ are each independently alkyl, aralkyl, or aryl;
R₃ and R₄ are each independently H or alkyl; and
n is 1, 2, 3, 4, 5, 6, 7, 8, 9, or 10.

In yet another aspect, the present disclosure provides methods of synthesizing a chemoproteomic capture reagent comprising:
contacting a solid support with an enrichment handle, thereby creating a solid support-enrichment handle conjugate;
contacting the solid support-enrichment handle conjugate with a solid-phase compatible cleavable linker, thereby creating a solid support-enrichment handle-solid-phase compatible cleavable linker conjugate;
contacting the solid support-enrichment handle-solid-phase compatible cleavable linker conjugate with a click capture amino acid, thereby creating a solid support-enrichment handle-solid-phase compatible cleavable linker-click capture amino acid conjugate;
contacting the solid support-enrichment handle-solid-phase compatible cleavable linker-click capture amino acid conjugate with an isotopically labelled amino acid, thereby creating a solid support-enrichment handle-solid-phase compatible cleavable linker-click capture amino acid-isotopically labelled amino acid conjugate; and
cleaving the support-enrichment handle-solid-phase compatible cleavable linker-isotopically labelled amino acid conjugate from the solid support, thereby synthesizing the chemoproteomic capture reagent.

In another aspect, the present disclosure provides methods of identifying a binding site comprising:
contacting substrate with an alkyne, thereby creating a substrate-alkyne conjugate;
contacting the substrate-alkyne conjugate with the chemoproteomic capture reagent disclosed herein, thereby creating a chemoproteomic capture reagent-substrate conjugate;
digesting the chemoproteomic capture reagent-substrate conjugate, thereby creating a digested substrate-chemoproteomic capture reagent conjugate;
contacting the digested substrate-chemoproteomic capture reagent conjugate with an enrichment agent;
cleaving the digested substrate-chemoproteomic capture reagent conjugate, thereby creating a digested substrate-isotopically labelled amino acid conjugate; and
determining the molecular weight of the digested substrate-isotopically labelled amino acid conjugate, thereby identifying the binding site.

### BRIEF DESCRIPTION OF THE DRAWINGS

FIG. 1A shows that solid phase peptide synthesis (SPPS) enables the synthesis of chemoproteomic capture reagents.

FIG. 1B shows prior approaches to prepare DADPS containing chemoproteomic capture reagents.

FIG. 1C shows the disclosed herein utilizing a solid-phase compatible DADPS reagent for high yielding synthesis of isotopically labeled chemically cleavable chemoproteomics capture reagents that enable quantitative chemoproteomic identification of cysteines accessible to modification by cysteine-reactive small molecules.

FIG. 2 shows the solid phase workflow for the synthesis of DADPS chemoproteomic capture reagents using NBIV-044 and NBIV-053.

FIG. 3 shows a cysteine profiling workflow resulting identified PSMs, unique peptides, and proteins identified by samples prepared using IAA and DADPS azide capture reagents NBIV-009, NBIV-011, NBIV-022, and NBIV-027, together with biotin-azide.

FIGS. 4A & 4B show the competitive ABPP workflow for identification of cysteines labeled by cysteines-reactive compounds using DADPS reagents NBIV-009 and NBIV-010, with structures shown in ‘B.’

FIG. 4C shows the distribution of Log 2 ratios for all unique peptides identified from competitive ABPP experiment following the workflow shown in ‘A’ using HEK293T lysates subjected to 500 μM KB02. Samples were prepared in triplicated and ratios>2 indicate peptides that harbor cysteines significantly modified by KB02.

FIG. 4D shows unique and overlapping cysteines (left) and KB02-labeled cysteines (Log₂ ratio>2; right) identified in samples prepared using DADPS reagents NBIV-009 and NBIV-010 compared with samples prepared using heavy and light biotin azide capture reagents.

FIG. 4E shows the median ratios and 95% confidence interval for samples prepared and analyzed using 1:1 and 4:1 DADPS capture reagents.

FIG. 5 shows an exemplary procedure for the solid-phase synthesis of peptides.

### DETAILED DESCRIPTION OF THE INVENTION

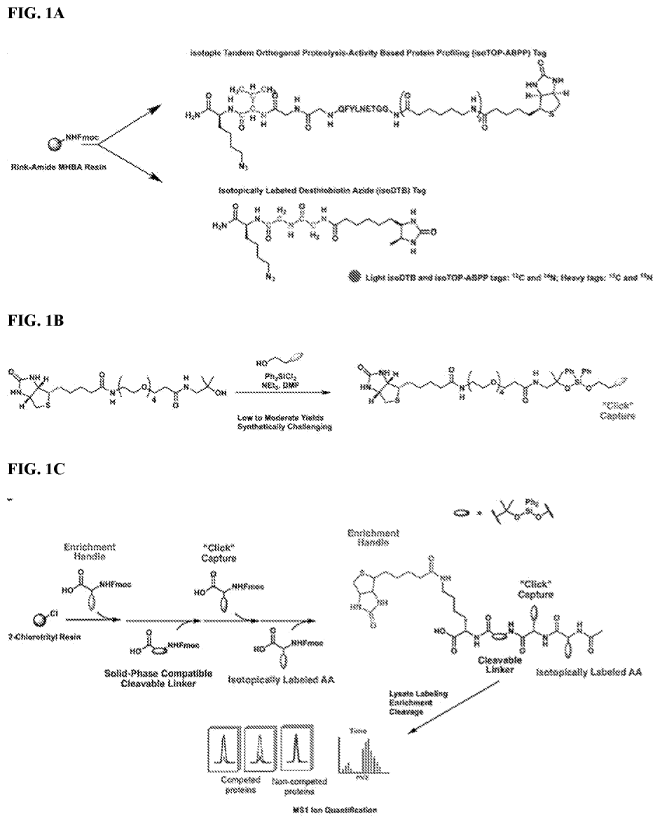

Solid-phase synthesis (SPS) has enabled the rapid and high yielding synthesis of peptides, proteins, oligonucleotides, and small molecule libraries. Given the high cost of isotopically labeled building blocks that enable quantitative chemoproteomics experiments, many isotopically labeled chemoproteomics capture reagents are often obtained through solid phase routes, which benefit from the near quantitative yields, ease of purification and facile incorporation of heavy isotopes. For example, the widely used TEV-cleavable capture reagents used in the isotopic tandem orthogonal proteolysis activity-based protein profiling (isoTOP-ABPP) workflow and the more recent isoDTB reagents are synthesized through SPPS (FIG. 1A).

The widely utilized isoTOP-ABPP method, while highly useful, does suffer from some limitations, including incompatibility with alternative sequence specific proteases (e.g. GluC and AspN that enable identification of additional cysteines) and contamination of samples with residual TEV protease peptides, which can result in decreased LC-MS/MS performance due to capillary blockage and spectral interference. Collectively, there is an unmet need for the production of reagents for chemoproteomic sample capture that fulfill the following criteria: (1) reagents obtained via SPPS, (2) incorporation of a chemically cleavable linker that is efficiently cleaved under mild conditions, (3) compatibility with all sequence specific proteases, and (4) high coverage of identified peptides.

Reagents that incorporate the dialkoxydiphenylsilane (DADPS) group fulfill all of these criteria. This linkage is cleaved under mild and MS-compatible acidic conditions (2-10% formic acid) and has shown to have superior protein and peptide coverage compared to diazobenzene linkers, a reductive cleavable linkage, as well as superior enrichment efficiency to other commonly employed cleavable linkers. It is anticipated that DADPS reagents should also prove compatible with all sequence specific proteases, although this compatibility remains unexplored. Despite their favorable properties for chemoproteomics, synthetic strategies for incorporation of DADPS moieties into enrichment reagents remain limited, with previously reported reagents requiring multi-step routes that are hindered by the often challenging and inefficient reactions required to form the DADPS linkage (FIG. 1B). These low yielding synthetic routes are particularly problematic for the synthesis of isotopically labeled reagents. Consequently, only a handful of such reagents have been reported, which were obtained in low overall yields.

This disclosure combines the advantages of the DADPS cleavable linker with the high yield and high throughput nature of solid phase synthesis to enable the rapid and combinatorial synthesis of DADPS chemoproteomics enrichment reagents. Through comparison of multiple synthetic strategies, a high yielding route to obtain two versatile fluorenylmethyl carbamate (Fmoc) functionalized building blocks (DADPS-Fmoc reagents) was found and these regents function analogously to Fmoc-protected amino acids commonly utilized in solid phase peptide synthesis (SPPS). Showing the utility of the innovative DADPS-Fmoc reagents, a panel of chemically cleavable chemoproteomics capture reagents was obtained in high yield and purity via SPSS, including isotopically differentiated (“light” and “heavy”) reagents, which were obtained via late-stage incorporation of a commercially available isotopically enriched amino acid (FIG. 1C). Application of these reagents to chemoproteomics analysis of the cysteinome identified >10,000 unique cysteine containing peptides. Using a DADPS-enabled competitive ABPP workflow with scout fragment KB02, 404 ligandable cysteines was identified and are expected to function as starting points for future covalent ligand discovery campaigns.

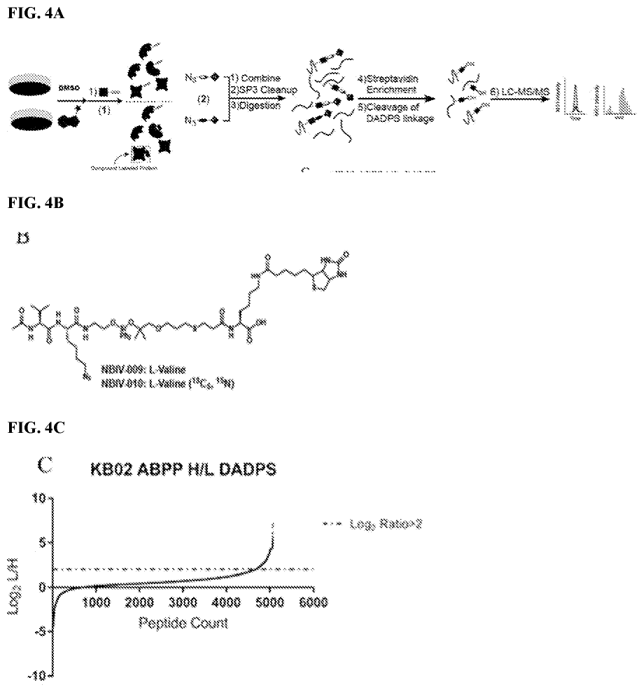

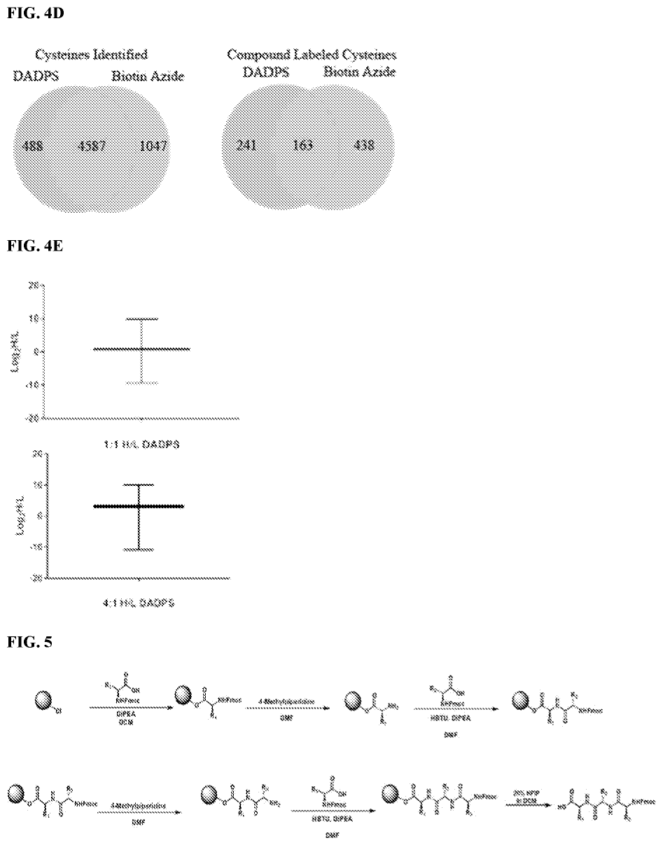

Disclosed herein are versatile and high yielding synthesis of SPS-compatible DADPS-FMOC building blocks NBIV-044 and NBIV-053, synthesis of which was enabled by a key high yielding thiol ene reaction. Demonstrating the synthetic utility of the reagents, application of the DADPS-FMOC reagent NBIV-044, which features a stable ether linkage, enabled the synthesis of a panel of chemoproteomic capture reagents that feature a biotin enrichment handle, azide moiety for click conjugation, DADPS cleavable linker for release from streptavidin resin and optionally an isotopic handle for MS1-based quantification that enables detection of compound-induced changes indicative of cysteine modification by small druglike molecules. Application of this reagent to proof-of-concept chemoproteomic studies identified 404 cysteines labeled by scout fragment KB02. The identified cysteines include previously identified and novel sites of modification. Benchmarking against previously reported biotin-azide based chemoproteomic capture reagents revealed comparable performance as indicated by similar numbers of PSMs, peptides and proteins identified by both reagents (FIG. 3) and a high degree of overlap between the cysteines identified by both methods (FIG. 4). As a number of cysteine-reactive compounds are currently FDA approved and in clinical trials, the identification of cysteines amenable to labeling or ‘drugging’ using chemoproteomics strategies is of high clinical relevance.

These data support the utility of the DADPS-FMOC reagent in the synthesis of high yielding and high performing chemoproteomics capture reagents, including those reported here as well as for future studies that require facile incorporation of additional useful features, such as alternative biorthogonal handles (e.g., for copper free click chemistry) or for incorporation of additional isotopic labels to move beyond two-plex multiplexing. Such isotopic labeling strategies should enable 3-plex MS1-based quantification through the use of “light,” “medium” and “heavy” valine reagents, analogous to 3-plex SILAC. Comparable to isobaric reagents, such as TMT and ITRAQ, that have enabled higher order multiplexing of samples, through MS2-based quantification, it is also envisioned that the utility of the DADPS-Fmoc building blocks will support the synthesis of isobaric chemoproteomic capture reagents. Such reagents will increase the throughput and reproducibility of chemoproteomics sample preparation and data acquisition. Given the widespread enthusiasm for chemoproteomics methods both in functional biology and drug development, it is expected that the aforementioned novel tools will be widely adopted in chemoproteomics studies, including those pursued in both academic and industrial settings.

Beyond chemoproteomics, it is expected that the DADPS-Fmoc reagents will also prove useful in other SPPS applications, including in the synthesis of peptides and proteins, which require incorporation of a cleavable moiety within their sequence. Collectively, the DADPS-FMOC reagent reported here is a highly versatile building block meritorious of future commercialization.

In one aspect, the present disclosure provides compounds of formula I or a salt thereof.

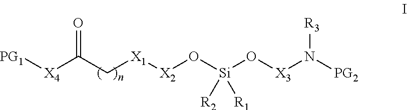

wherein,
X₁ and X₄ are each independently O, S, or NR₄;
X₂ and X₃ are each independently alkylene;
PG₁ is H, an oxygen protecting group, or a sequence of amino acids;
PG₂ is H, a nitrogen protecting group or a sequence of amino acids; and
R₁ and R₂ are each independently alkyl, aralkyl, or aryl;
R₃ and R₄ are each independently H or alkyl; and
n is 1, 2, 3, 4, 5, 6, 7, 8, 9, or 10.

In certain preferred embodiments, X₁ is S.

In certain preferred embodiments, X₂ is alkyloxyalkyl. In certain embodiments, X₂ is substituted with alkyl, alkenyl, alkynyl, ester, amido, aryl, or heteroaryl. In certain preferred embodiments X₂ is substituted with alkyl (e.g., methyl).

In certain embodiments, X₃ is methyl, ethyl, propyl, butyl, pentyl, hexyl, heptyl, or octyl. In certain embodiments X₃ is hexyl. In other embodiments X₃ is propyl. In certain embodiments, X₃ is substituted with alkyl, alkenyl, alkynyl, ester, amido, aryl, or heteroaryl.

In certain embodiments, R₃ is H.

In certain preferred embodiments, n is 2.

In certain preferred embodiments, X₄ is O. In other embodiments, X₄ is NR₄. In some embodiments, R₄ is H.

In certain embodiments, PG₁ is alkyl, benzyl, or heteroaryl. In certain preferred embodiments, PG₁ is H. In certain embodiments, PG₁ is a sequence of amino acids (e.g., 1-10 amino acids).

In certain embodiments, the compound has a structure represented by formula Ia or a salt thereof:

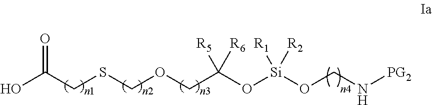

wherein,
n1, n2, n3, and n4 are each independently 1, 2, 3, 4, 5, 6, 7, 8, 9, or 10;
R₅ and R₆ are each independently alkyl, alkenyl, alkynyl, ester, amido, aryl, or heteroaryl.

In certain preferred embodiments, n1 is 2. In certain preferred embodiments, n2 is 3. In certain preferred embodiments, n3 is 1. In certain embodiments, n4 is 2. In other embodiments, n4 is 6. In certain preferred embodiments, R₅ is alkyl (e.g., methyl). In certain preferred embodiments, R₆ is alkyl (e.g., methyl). In certain preferred embodiments, R₁ is aryl (e.g., phenyl). In certain preferred embodiments, R₂ is aryl (e.g., phenyl). In certain embodiments, PG₂ is alkyl, arakyl, carbamyl, heteroaryl, hetercyclyl, acetyl, or sulfonyl. In other embodiments, PG₂ is Fmoc. In other embodiments, PG₂ is sequence of amino acids (e.g., 1-10 amino acids).

In certain preferred embodiments, compound is selected from:

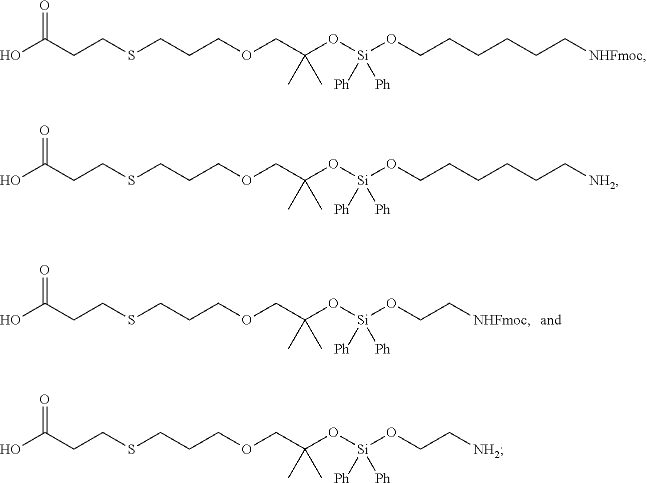

a salt thereof.

In yet another aspect, the present disclosure provides methods of synthesizing a chemoproteomic capture reagent comprising:
contacting a solid support with an enrichment handle, thereby creating a solid support-enrichment handle conjugate;
contacting the solid support-enrichment handle conjugate with a solid-phase compatible cleavable linker, thereby creating a solid support-enrichment handle-solid-phase compatible cleavable linker conjugate;
contacting the solid support-enrichment handle-solid-phase compatible cleavable linker conjugate with a click capture amino acid, thereby creating a solid support-enrichment handle-solid-phase compatible cleavable linker-click capture amino acid conjugate;
contacting the solid support-enrichment handle-solid-phase compatible cleavable linker-click capture amino acid conjugate with an isotopically labelled amino acid, thereby creating a solid support-enrichment handle-solid-phase compatible cleavable linker-click capture amino acid-isotopically labelled amino acid conjugate; and
cleaving the support-enrichment handle-solid-phase compatible cleavable linker-isotopically labelled amino acid conjugate from the solid support, thereby synthesizing the chemoproteomic capture reagent.

In certain embodiments, the solid support is a resin. In certain preferred embodiments, the resin is a chlorotrityl resin.

In certain preferred embodiments, the enrichment handle is an amino acid substituted with biotin (e.g., an amino acid having a side chain substituted with biotin). In certain embodiments, the amino acid is a naturally occurring amino acid (e.g., lysine or cysteine).

In certain preferred embodiments, the solid-phase compatible cleavable linker is the chemoproteomic capture reagent disclosed herein.

In certain preferred embodiments, the click capture amino acid is an azide-containing amino acid (e.g., an amino acid having a side chain substituted with an azide).

In certain embodiments, the isotopically labelled amino acid is a naturally occurring amino acid (e.g., valine or alanine). In certain preferred embodiments, the isotopically labelled amino acid is enriched with C¹³ or N¹⁵.

In certain preferred embodiments, the support-enrichment handle-solid-phase compatible cleavable linker-isotopically labelled amino acid conjugate is cleaved from the solid support using acid (e.g., hydrochloric acid).

In another aspect, the present disclosure provides methods of identifying a binding site comprising:
contacting substrate with an alkyne, thereby creating a substrate-alkyne conjugate;
contacting the substrate-alkyne conjugate with the chemoproteomic capture reagent disclosed herein, thereby creating a chemoproteomic capture reagent-substrate conjugate;
digesting the chemoproteomic capture reagent-substrate conjugate, thereby creating a digested substrate-chemoproteomic capture reagent conjugate;
contacting the digested substrate-chemoproteomic capture reagent conjugate with an enrichment agent;
cleaving the digested substrate-chemoproteomic capture reagent conjugate, thereby creating a digested substrate-isotopically labelled amino acid conjugate; and
determining the molecular weight of the digested substrate-isotopically labelled amino acid conjugate, thereby identifying the binding site.

In certain embodiments, the substrate is a protein. In certain embodiments, the protein is formed from cell lysation.

In certain preferred embodiments, the method comprises contacting the substrate-alkyne conjugate with the chemoproteomic capture reagent disclosed herein, forming a triazole linking the chemoproteomic capture reagent to the substrate.

In certain preferred embodiments, the method comprises digesting the chemoproteomic capture reagent-substrate conjugate comprising contacting the chemoproteomic capture reagent-substrate conjugate with a digestion enzyme (e.g., trypsin).

In certain preferred embodiments, the enrichment agent is a protein that binds biotin (e.g., avidin or streptavidin).

In certain preferred embodiments, the method comprises cleaving the digested substrate-chemoproteomic capture reagent conjugate comprising contacting the digested substrate-chemoproteomic capture reagent conjugate with acid (e.g., formic acid).

This disclosure also includes all suitable isotopic variations of a compound or chemoproteomic capture reagent of the disclosure. An isotopic variation of a compound or chemoproteomic capture reagent of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulfur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I, respectively. Accordingly, recitation of “hydrogen” or “H” should be understood to encompass ¹H (protium), ²H (deuterium), and ³H (tritium) unless otherwise specified. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Such variants may also have advantageous optical properties arising, for example, from changes to vibrational modes due to the heavier isotope. Isotopic variations of a compound of the invention can generally be prepared by conventional procedures known by a person skilled in the art such as by the illustrative methods or by the preparations described in the examples hereafter using appropriate isotopic variations of suitable reagents.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amido”, as used herein, refers to a group

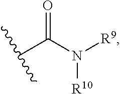

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

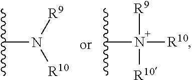

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

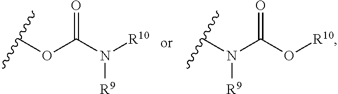

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring.

Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “cycloalkyl” includes substituted or unsubstituted non-aromatic single ring structures, preferably 4- to 8-membered rings, more preferably 4- to 6-membered rings. The term “cycloalkyl” also includes polycyclic ring systems having two or more cyclic rings in which two or more carbons are common to two adjoining rings wherein at least one of the rings is cycloalkyl and the substituent (e.g., R¹⁰⁰) is attached to the cycloalkyl ring, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, pyrimidine, denzodioxane, tetrahydroquinoline, and the like.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms.

Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamido” is art-recognized and refers to the group represented by the general formulae

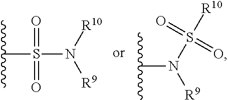

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

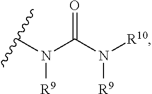

wherein R⁹ and R¹⁰ independently represent hydrogen or a hydrocarbyl.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

### EXAMPLES

The invention now being generally described, it will be more readily understood by reference to the following examples, which are included merely for purposes of illustration of certain aspects and embodiments of the present invention, and are not intended to limit the invention.

### Example 1: Synthesis of Exemplary Chemoproteomics Capture Reagents

### Establishing a Route to a Prototype DADPS Building Block.

While the utility of silyl groups for capture and release of cargo on the solid phase was previously demonstrated, the feasibility of DADPS group incorporation into polypeptides obtained through SPPS remains unexplored. Therefore, the first step was to develop a synthetic route to enable high yielding incorporation of the DADPS group into a range of peptide-based reagents. It was initially envisioned that the prototype DADPS reagent containing a free carboxylic acid for solid phase coupling could easily be obtained by reacting β-hydroxyisovaleric acid with N-cbz-1-amino-6-hexanol (Scheme S1). However, under all reaction conditions tested, none of the desired product was observed and instead observed homo coupling of the primary alcohol was found. It was speculated that the free acid was likely not compatible with the basic conditions required for DADPS formation. Therefore, the next step was to generate ester protected substrates to assess the DADPS formation in the absence of the free acid moiety.

A benzyl carbamate (Cbz) protected amino alcohol was used as part of the initial investigation of the DADPS formation reaction indicated that the addition of 4-dimethylaminopyridine (DMAP) afforded increased yields, which rendered Fmoc-protected reagents incompatible with the basic DADPS formation conditions (Scheme S2). A panel of six prototype ester-based DADPS reagents were readily obtained upon condensation with Cbz protected aminohexanol 1, in yields ranging from 43 to 85% (Scheme S3). All attempts at hydrolysis of the DADPS ester moieties afforded only undesired Cbz deprotection or cleavage of the silane (Table S1). Silane cleavage predominated for cleavage conditions that required strong Lewis acids, which was ascribed to coordination of the silyl diether oxygens facilitating cleavage.

As no desired product was observed for any of the ester substrates, it was next evaluated whether a DADPS reagent that featured an activated ester could be both obtained and subjected to the necessary protecting group manipulations to afford the desired Fmoc protected DADPS activated ester. This strategy would obviate the need for hydrolysis as one could directly couple the activated ester onto solid phase through amide bond formation. Following established conditions, five different activated ester analogues of beta-hydroxy isovaleric acid (Scheme S4) were obtained in near quantitative yields (78-99%) and subjected those to DADPS formation conditions. In all instances the desired product was not obtained in any appreciable yield. In reactions utilizing the N-hydroxysuccinimide (NHS) and 2,3,5,6-tetrafluorophenol (TFP) esters, by LC-MS observed displacement of the activated ester (Scheme S5) was observed, indicating in situ formation of the product followed by reaction of the ester with the Cbz-amino alcohol. Changing the base from DMAP to TEA did afford conversion to the desired product, as detected by MS (Scheme S6). However, the prior observation that DMAP was required for high yield formation of DADPS reagents combined with the general observed instability of the activated ester building blocks tempered enthusiasm for this route.

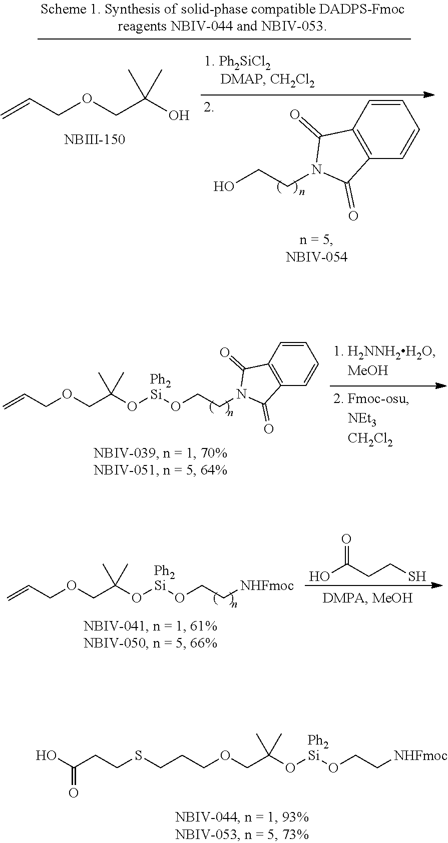

### Synthesis and Validation of Solid Phase Compatibility of the FMOC-DADPS-Acids NBIV-044 and NBIV-053

Given the generally mild and orthogonal conditions required for thiol-ene chemistry and the availability of the allyl ester model substrate 2, it was tested whether 2 could be coupled to 3-mercaptopropionic acid (MPA) under photoinitiated reaction conditions. Gratifyingly, product formation in 61% yield (Scheme S7A) was observed. However, all efforts towards selective deprotection of the Cbz group in the presence of the allyl ester were unproductive (Scheme S7B). The Cbz group was thus replaced with phthalimide protected amine 3, which afforded allyl ester DADPS-Fmoc reagent 4 in 92% yield. Subsequent protecting group manipulation afforded Fmoc protected DADPS reagent 5 in 70% yield over two steps.

Compound 5 was then subjected to a photoinitiated thiol-ene reaction with MPA to form a thioether linkage and free carboxylic acid on the reagent, obtaining the solid phase compatible DADPS reagent 6 in 59% yield (Scheme S8). While the high yield formation of this model substrate was encouraging, a decision was made to modify the strategy to eliminate the ester moiety, due to its potential hydrolytic instability in esterase-containing cell lysates. Additionally, there was a concern about the aqueous media solubility of the reagent bearing the long hexyl chain. Accordingly, the sequence of DADPS formation was repeated with an allyl ether and making two reagents NBIV-044 and NBIV-053., which differed by alkyl chain length. Notably, the thiol-ene was performed neat, providing the final solid-phase compatible reagent in 39% yield for the reagent bearing an ethyl chain NBIV-044 and 31% yield for the hexyl chain NBIV-053 over 4 steps.

### SPS and Proteomic Benchmarking of a Panel of DADPS Functionalized Chemoproteomics Capture Reagents NBIV-009, NBIV-011, NBIV-022, and NBIV-027.

With a working strategy for DADPS enrichment reagent synthesis in hand, the next step was to synthesize a panel of reagents. Three variables were explored, the linker length, the source of azide, and type of amino acid used for isotopic labeling reagent synthesis. The first to be prioritized linker length as the inventors wanted to assess how changes to the reagent size and solubility would impact coverage. For azide source, ß-azidohomoalanine was compared with azidolysine with the goal of again determining how reducing the reagent size would impact proteomic coverage. It was speculated that there could differences in the fragmentation pattern of azidohomoalanine and azidolysine based reagents, which could impact coverage of chemoproteomics detected peptides. Lastly, given the ready availability of various isotopically labeled amino acids, most notably valine and alanine, it was sought to assess whether incorporation of isotopically labeled amino acids would enable MS1-based quantification of enriched peptides and whether amino acid selection would impact reagent performance.

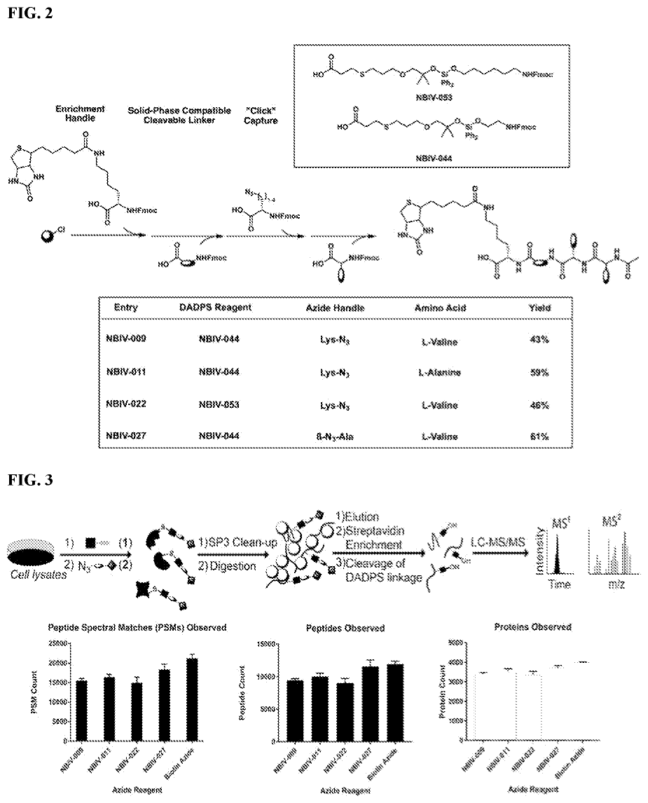

With these objectives in mind, a panel of 4 reagents (NBIV-009, NBIV-011, NBIV-022, and NBIV-027) were synthesized in high yield and purity, (FIG. 2) with the goal of systematically comparing each of the aforementioned variables. Using HEK293T cell lysates, cysteine-containing peptides were captured and identified, using a modified version of the SP3 workflow for analysis of the cysteinome (FIG. 3). First cysteines were capped with the highly reactive cysteine alkylating reagent iodoacetamide alkyne (IAA). The alkyne-labeled lysates were then subjected to click conditions with each of the azido-DADPS capture reagents followed by SP3 sample cleanup, tryptic digest, capture of labeled peptides with streptavidin, followed by release of DADPS labeled peptides under mild acidic conditions. LC-MS/MS analysis revealed similar performance for all reagents, as indicated by the comparable numbers of PSMs, peptides, and protein identifications (FIG. 3). Similar performance for the alanine and valine reagents NBIV-009 and NBIV-011 was observed, indicating that the synthesis of isotopically labeled DADPS reagents should proceed smoothly using either heavy valine or heavy alanine building blocks. The use of neutravidin for samples prepared with biotin-azide in place of streptavidin resin for samples prepared with the DADPS reagents Slight differences in coverage and peptides identified using biotin-azide vs DAPDS reagents can be rationalized by the use of neutravidin vs streptavidin resin for the respective workflows.

### MS1-Based Quantification to Discover Ligandable Cysteines Using Isotopically Differentiated Reagents NBIV-009 and NBIV-010.

To obtain an isotopically enriched DADPS capture reagent, the synthesis and application of heavy L-valine (¹³C₅ ¹⁵N)— containing reagent NBIV-010 was focused on, as the relatively large +6 Da mass difference is ideal for MS1-based quantification. Further motivating the isotopic reagent design, a +6-mass difference is used in isoTOP-ABPP and isoDTB reagents together with the previously reported heavy and light azido-biotin reagents, which was envisioned could facilitate head-to-head comparisons. Using the same synthetic strategy, heavy reagent NBIV-010 was obtained in 58% yield. LC-MS analysis revealed comparable intensities of light and heavy reagents, when assayed as a 1:1 mixture. These reagents in a were further validated in a competitive ABPP workflow. Using HEK293T cell lysates, samples were subjected to either vehicle, DMSO, or 500 μM KB02. Upon labeling with IAA and clicking the samples with either heavy or light DADPS probe (NBIV-010 or NBIV-009, respectively; FIG. 4B) or heavy or light biotin azide, the samples were subjected to SP3 cleanup. After enrichment with either streptavidin resin, for DADPS labeled peptides, or NeutrAvidin resin, for biotin azide labeled peptides, the peptides were cleaved or eluted off resin, respectively, and analyzed by LC-MS/MS. Across three biological replicates, the DADPS reagents identified 5075 unique cysteines, with 404 found in peptides with Log₂MS1 extracted ion chromatograph area ratios>2 (FIG. 4C)—these elevated ratios indicate cysteines that are modified by KB02. Comparable coverage and ratios were observed for samples prepared using heavy/light biotin-azide reagents, and 75% of identified cysteines were shared across both reagent datasets (FIG. 4D, left panel). Demonstrating the utility of assaying multiple capture reagents, 488 cysteines were uniquely identified by the DADPS capture reagents. The DADPS and biotin azide datasets shared 163 cysteines with ratios>2, which represent a high confidence dataset of KB02-labeled sites (FIG. 4D, right panel). The performance of the DADPS capture reagents was further vetted using reagent dilution experiments to generate datasets with expected Log₂ ratios near zero, for 1:1 samples and near two for 4:1 samples (FIG. 4E).

### Chemistry Methods

### General Methods

All reactions were performed in dried glassware under an atmosphere of dry N₂ unless otherwise stated. Silica gel P60 (SiliCycle) was used for column chromatography. Plates were visualized by fluorescence quenching under UV light or by staining with iodine, KMnO₄, or bromocresol green. Other reagents were purchased from Sigma-Aldrich (St. Louis, MO), Alfa Aesar (Ward Hill, MA), EMD Millipore (Billerica, MA), Fisher Scientific (Hampton, NH), Oakwood Chemical (West Columbia, SC), Combi-blocks (San Diego, CA) and Cayman Chemical (Ann Arbor, MI) and used without further purification. ¹H NMR and ¹³C NMR spectra for characterization of new compounds and monitoring reactions were collected in CDCl₃, CD₃OD, CD₆CO or DMSO-d₆ (Cambridge Isotope Laboratories, Cambridge, MA) on a Bruker AV 500 MHz spectrometer or Brucker AV 400 MHz in the Department of Chemistry & Biochemistry at The University of California, Los Angeles. All chemical shifts are reported in the standard notation of parts per million using the peak of residual proton signals of the deuterated solvent as an internal reference. Coupling constant units are in Hertz (Hz). Splitting patterns are indicated as follows: br, broad; s, singlet; d, doublet; t, triplet; q, quartet; m, multiplet; dd, doublet of doublets; dt, doublet of triplets. Low-resolution mass spectrometry was performed on an Agilent Technologies InfinitiyLab LC/MSD single quadrupole LC/MS (ESI source). High-resolution mass spectrometry was performed on a Waters LCT Premier with ACQUITY LC and autosampler (ESI source). Cell culture reagents including Dulbecco's phosphate-buffered saline (DPBS), Dulbecco's modified Eagle's medium (DMEM)/high glucose media, Roswell Park Memorial Institute (RPMI) media, trypsin-EDTA and penicillin/streptomycin (Pen/Strep) were purchased from Fisher Scientific. All protein concentrations were determined using a Bio-Rad DC protein assay kit using reagents from Bio-Rad Life Science (Hercules, CA).

### 1-(Allyloxy)-2-Methylpropan-2-ol

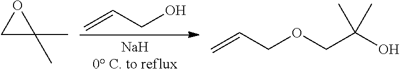

To a 100 mL round-bottom flask was added allyl alcohol (10.2 g, 12.0 mL, 3 Eq, 176 mmol) and cooled to 0° C. To this solution was slowly added sodium hydride (2.4 g, 60% Wt, 1.0 Eq, 58.8 mmol) and then let stir at 0° C. for 20 min. Next, 2,2-dimethyloxirane (4.24 g, 5.22 mL, 1 Eq, 58.8 mmol) was added and solution refluxed at 52° C. Upon completion of reaction as determined by TLC (3 hours) the reaction mixture was diluted with sat. NH₄Cl and extracted with Et₂O (3×30 mL). Combined organic layers were dried over sodium sulfate and volatiles removed under reduced pressure. The crude residue was purified by vacuum distillation yielding the desired alcohol as a clear liquid (5.18 g, 67.7%). All analyses were consistent with previously reported data.^{2 1}H NMR (400 MHz, CDCl₃) δ 5.96-5.82 (m, 1H), 5.30-5.14 (m, 2H), 4.02 (ddt, J=5.1, 3.2, 1.4 Hz, 2H), 3.26 (d, J=3.1 Hz, 2H), 1.20 (d, J=3.4 Hz, 6H).

### Benzyl (6-Hydroxyhexyl)carbamate

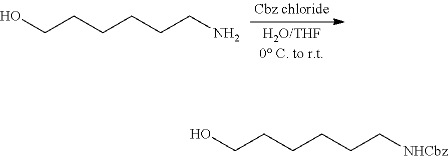 [SMILES: NCCCCCCO ; O=C(NCCCCCCO)OCc1ccccc1]

To a 250 mL round-bottom flask was added 6-aminohexan-1-ol (2.985 g, 1 Eq, 24.71 mmol), sodium carbonate (5.761 g, 2.2 Eq, 54.36 mmol), Water (35 mL), and THF (35 mL). The flask was purged with argon, cooled to 0° C., and benzyl chloroformate (4.636 g, 3.880 mL, 1.1 Eq, 27.18 mmol) added dropwise over 5 min. Solution was then let warm to room temperature overnight. Upon completion, the reaction mixture was diluted with water and extracted with ethyl acetate (3×40 mL). Combined organic layers dried over sodium sulfate and solvent removed under reduced pressure to yield the desired product as a white solid (4.5 g, 72%). All analyses were consistent with previously reported data.^{3 1}H NMR (400 MHz, CDCl₃) δ 7.40-7.29 (m, 5H), 5.09 (s, 2H), 3.63 (q, J=6.3 Hz, 2H), 3.20 (q, J=6.7 Hz, 2H), 1.53 (ddt, J=20.4, 13.8, 6.6 Hz, 4H), 1.43-1.29 (m, 4H).

### 2-(6-Hydroxyhexyl)isoindoline-1,3-dione

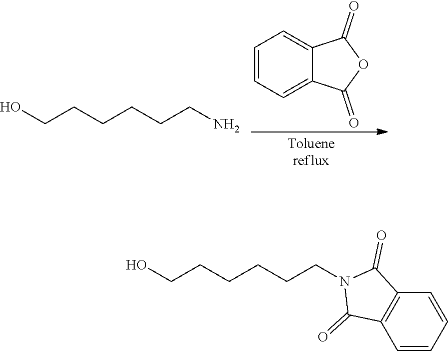 [SMILES: NCCCCCCO ; O=C1OC(=O)c2ccccc21 ; O=C1c2ccccc2C(=O)N1CCCCCCO]

6-aminohexan-1-ol (2.00 g, 1 Eq, 17.1 mmol) and phthalic anhydride (2.53 g, 1 Eq, 17.1 mmol) dissolved in Toluene (50 mL) were refluxed with a dean-stark trap. After the reaction was judged complete by TLC (2 hours) the reaction mixture was cooled to room temperature and volatiles removed under reduced pressure. Crude material was then purified by silica column chromatography (1:1 to 2:1 ethyl acetate:hexanes) to yield the desired product as a white crystalline solid (3.9 g, 92%). All analyses were consistent with previously reported data. ¹H NMR (400 MHz, CDCl₃) δ 7.82 (dd, J=5.4, 3.1 Hz, 2H), 7.69 (dd, J=5.4, 3.0 Hz, 2H), 3.69-3.64 (m, 2H), 3.61 (t, J=6.5 Hz, 2H), 1.77-1.61 (m, 3H), 1.60-1.50 (m, 2H), 1.45-1.29 (m, 3H).

### Benzyl (6-((((2-methyl-1-phenylpropan-2-yl)oxy)diphenylsilyl)oxy)hexyl)carbamate

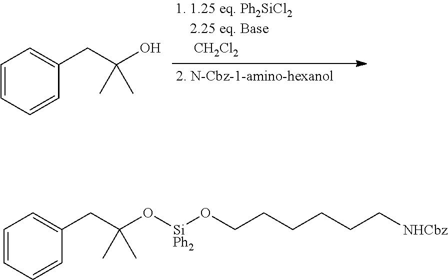

| Entry # | Base | Temperature (1st Step) | Yield |
|---|---|---|---|
| 1 | DMAP | 0° C. to 25° C. | 59% |
| 2 | NEt₃ | 0° C. to 25° C. | 18% |
| 3 | NEt₃ | 0° C. to 40° C. | 22% |

To an oven dried 50 mL round-bottom flask was added 2-methyl-1-phenylpropan-2-ol (150 mg, 154 μL, 1 Eq, 1.00 mmol) and Base (2.25 Eq, 2.25 mmol). The flask was capped with rubber septa and purged with argon followed by addition of anhydrous DCM (5.00 mL). The solution was then cooled to 0° C. and diphenyldichlorosilane (317 mg, 257 μL, 1.25 Eq, 1.25 mmol) was added dropwise. After complete addition the reaction was allowed to warm to specified temperature and stir for 5 hours. After 5 hours the solution was then cooled back to 0° C. and benzyl (6-hydroxyhexyl)carbamate (264 mg, 1.05 Eq, 1.05 mmol) was added. The solution was then allowed to warm to room temperature and stir for 16 h. After completion, the reaction mixture was diluted with sat. sodium bicarbonate and extracted with CH₂Cl₂ (3×10 mL). Combined organic layers were washed with brine (1×10 mL) and dried over sodium sulfate. Crude product was purified by silica column chromatography (1:9 to 1:3 ethyl acetate:hexanes) to yield the pure product as a clear oil. ¹H NMR (400 MHz, CDCl₃) δ 7.60 (dt, J=8.0, 1.7 Hz, 4H), 7.42-7.30 (m, 11H), 7.28-7.21 (m, 5H), 5.10 (s, 2H), 3.60 (t, J=6.5 Hz, 2H), 3.16 (q, J=6.8 Hz, 2H), 2.85 (s, 2H), 1.48 (dt, J=18.1, 7.0 Hz, 4H), 1.37-1.28 (m, 2H), 1.25 (d, J=1.4 Hz, 6H). ¹³C NMR (101 MHz, CDCl₃) δ 156.49, 138.60, 136.81, 135.30, 135.13, 131.02, 129.89, 128.65, 128.27, 128.22, 127.84, 127.70, 126.23, 75.85, 66.72, 62.83, 51.20, 41.20, 32.32, 30.06, 29.78, 26.59, 25.58. HRMS (ESI-MS) m/z: Calculated [M+Na]⁺=604.2859, Found [M+Na]+=604.2869

### 2-(6,6-Dimethyl-8,8-diphenyl-4,7,9-trioxa-8-silapentadec-1-en-15-yl)isoindoline-1,3-dione

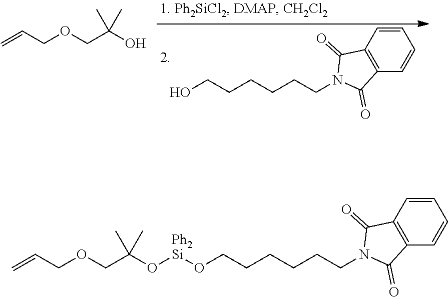

To an oven dried 250 mL round-bottom flask was added DMAP (2.228 g, 2.25 Eq, 18.23 mmol), 1-(allyloxy)-2-methylpropan-2-ol (1.055 g, 1 Eq, 8.104 mmol) and capped with septa. The system was purged with argon and CH₂Cl₂ (30.0 mL) was added. The solution was cooled to 0° C. and diphenyldichlorosilane (2.565 g, 2.085 mL, 1.25 Eq, 10.13 mmol) was added dropwise. The solution was then allowed to warm to room temperature. After the first step was determined to be complete by TLC (5 hours), the solution was cooled to 0° C. The flask was uncapped and 2-(6—hydroxyhexyl)isoindoline-1,3-dione (2.104 g, 1.05 Eq, 8.509 mmol) was added. The flask was recapped and the reaction was warmed to room temperature and stirred overnight. Upon completion the reaction was diluted with sat. sodium bicarbonate and extracted with CH₂Cl₂ (3×40 mL). Combined organic layers were washed with brine and dried over sodium sulfate. Crude product was purified by silica column chromatography (1:9 to 1:3 ethyl acetate:hexanes) to yield the pure product as a clear oil (2.9 g, 64%). ¹H NMR (400 MHz, CDCl₃) δ 7.84 (dd, J=5.4, 3.1 Hz, 2H), 7.70 (dd, J=5.5, 3.0 Hz, 2H), 7.66-7.61 (m, 3H), 7.40-7.27 (m, 7H), 5.85 (ddd, J=22.7, 10.6, 5.4 Hz, 1H), 5.27-5.09 (m, 3H), 3.93 (dt, J=5.4, 1.5 Hz, 2H), 3.72 (t, J=6.5 Hz, 2H), 3.65 (t, J=7.3 Hz, 2H), 3.29 (s, 2H), 1.65 (p, J=7.5 Hz, 2H), 1.59-1.52 (m, 5H), 1.44-1.29 (m, 2H), 1.27 (s, 5H). ¹³C NMR (101 MHz, CDCl₃) δ 168.56, 135.37 (d), 135.07, 134.89 (d), 133.96, 132.33, 129.88, 127.69, 123.28, 116.47, 79.22, 75.40, 72.41, 62.94, 38.15, 32.38, 28.76, 27.48, 26.80, 25.59. HRMS (ESI-MS) m/z: Calculated [M+Na]⁺=580.2495, Found [M+Na]⁺=580.2493

### (9H-Fluoren-9-yl)methyl (6,6-dimethyl-8,8-diphenyl-4,7,9-trioxa-8-silapentadec-1-en-15-yl)carbamate

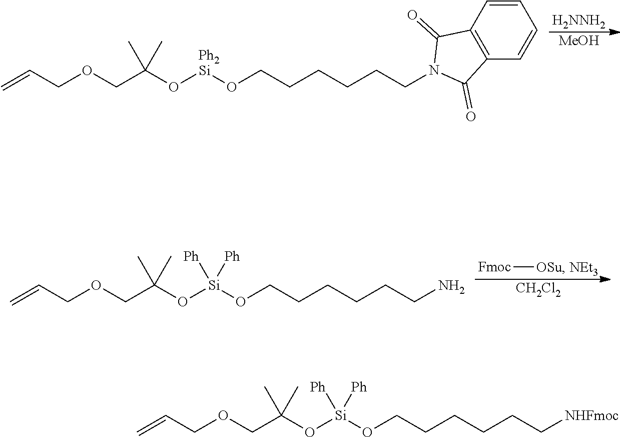

To an oven dried 100 mL round-bottom flask was added 2-(6,6-dimethyl-8,8-diphenyl-4,7,9-trioxa—8-silapentadec-1-en-15-yl)isoindoline-1,3-dione (2.890 g, 1 Eq, 5.181 mmol). The vial was capped and purged with nitrogen followed by addition of MeOH (25.91 mL) and then dropwise addition of hydrazine hydrate (1.038 g, 1.005 mL, 4 Eq, 20.73 mmol). The solution was left to stir at room temperature overnight. Upon completion, the reaction mixture was diluted with 1M sodium carbonate and 1M oxalic acid and extracted with ethyl acetate (3×30 mL). Then, the combined organic extracts were washed with brine and dried over sodium sulfate. Volatiles were removed under reduced pressure and material used in the next step without further purification. To an oven dried 100 mL round-bottom flask was added Fmoc-osu (2.097 g, 1.2 Eq, 6.218 mmol), capped, and purged with argon. The crude amine from the first step was dissolved in dry CH₂Cl₂ (30 mL) and added to this flask. Reaction mixture was then cooled to 0° C. and triethylamine (1.258 g, 1.73 mL, 2.4 Eq, 12.44 mmol) added. The solution was left to stir at room temperature for 16 hours. Upon completion, the reaction was diluted with water and extracted with CH₂Cl₂ (3×30 mL). Organic layers combined and washed with brine then dried over sodium sulfate. The crude material was purified by silica column chromatography (1:9 to 1:3 ethyl acetate:hexanes) to yield the desired product as a pale yellow oil (2.23 g, 66%). ¹H NMR (400 MHz, CDCl₃) δ 7.77 (d, J=7.5 Hz, 3H), 7.68-7.63 (m, 5H), 7.63-7.58 (m, 3H), 7.43-7.28 (m, 14H), 5.86 (ddt, J=17.3, 10.6, 5.4 Hz, 1H), 5.27-5.11 (m, 2H), 4.71 (s, 1H), 4.41 (d, J=6.9 Hz, 2H), 4.22 (t, J=6.9 Hz, 1H), 3.94 (dt, J=5.4, 1.5 Hz, 2H), 3.74 (t, J=6.5 Hz, 2H), 3.31 (s, 2H), 3.16 (q, J=6.9 Hz, 2H), 1.63-1.53 (m, 2H), 1.46 (q, J=7.6 Hz, 2H), 1.37 (td, J=13.4, 11.8, 6.1 Hz, 2H), 1.29 (s, 6H). ¹³C NMR (101 MHz, CDCl₃) δ 156.52, 144.17, 141.46, 135.27, 135.09, 134.90, 129.92, 127.70, 127.15, 125.16, 120.10, 116.50, 79.21, 75.42, 72.42, 62.93, 47.46, 41.19, 32.38, 30.09, 27.50, 26.62, 25.64. HRMS (ESI-MS) m/z: Calculated [M+Na]⁺=672.3121, Found [M+Na]⁺=672.3126.

### 1-(9H-Fluoren-9-yl)-14,14-dimethyl-3-oxo-12,12-diphenyl-2,11,13,16-tetraoxa-20-thia-4-aza-12-silatricosan-23-oic acid

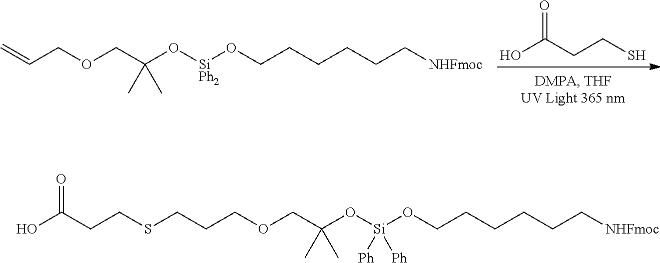

To an oven-dried 25 mL round-bottom flask was added 2,2-dimethoxy-2-phenylacetophenone (94.5 mg, 0.5 Eq, 369 μmol), 3-mercaptopropanoic acid (235 mg, 193 μL, 3 Eq, 2.21 mmol), and (9H-fluoren-9-yl)methyl (6,6-dimethyl-8,8-diphenyl-4,7,9-trioxa-8-silapentadec-1-en-15-yl)carbamate (500 mg, 1 Eq, 738 μmol) which were dissolved in dry THF (2.95 mL). The solution was then sparged with nitrogen for 10 minutes to remove oxygen. The reaction mixture was then allowed to stir at room temperature under UV irradiation (365 nm) until judged complete by TLC (4 hours). Upon completion the reaction mixture was diluted with sat. sodium bicarbonate and extracted with ethyl acetate (5×15 mL). The combined organic layers were washed with sat. ammonium chloride and brine then dried over sodium sulfate. The crude material was purified by silica column chromatography (1:1 to 100% ethyl acetate:hexanes) to yield the desired product as a pale yellow oil (360 mg, 62%). ¹H NMR (400 MHz, CDCl₃) δ 7.76 (d, J=7.5 Hz, 2H), 7.64 (dt, J=6.6, 1.6 Hz, 4H), 7.58 (d, J=7.5 Hz, 2H), 7.42-7.28 (m, 10H), 4.79 (br, 1H), 4.48-4.33 (m, 2H), 4.21 (t, J=6.9 Hz, 1H), 3.73 (t, J=6.5 Hz, 2H), 3.43 (t, J=6.0 Hz, 2H), 3.28 (d, J=2.6 Hz, 2H), 3.14 (dt, J=17.4, 8.7 Hz, 2H), 2.74 (s, 2H), 2.59 (q, J=8.3 Hz, 4H), 1.79 (t, J=6.8 Hz, 2H), 1.64-1.51 (m, 2H), 1.51-1.32 (m, 2H), 1.27 (m, 10H). ¹³C NMR (101 MHz, CDCl₃) δ 141.45, 135.04, 134.89, 134.84, 129.90, 127.80, 127.69, 127.15, 125.14, 120.09, 79.80, 77.36, 75.39, 69.75, 69.74, 66.66, 62.94, 47.42, 41.18, 34.69, 32.38, 29.80, 28.96, 27.45, 26.83, 26.58, 25.63. HRMS (ESI-MS) m/z: Calculated [M+Na]⁺=778.3210, Found [M+Na]⁺=778.3209

### 2-(6,6-Dimethyl-4,4-diphenyl-3,5,8-trioxa-4-silaundec-10-en-1-yl)isoindoline-1,3-dione

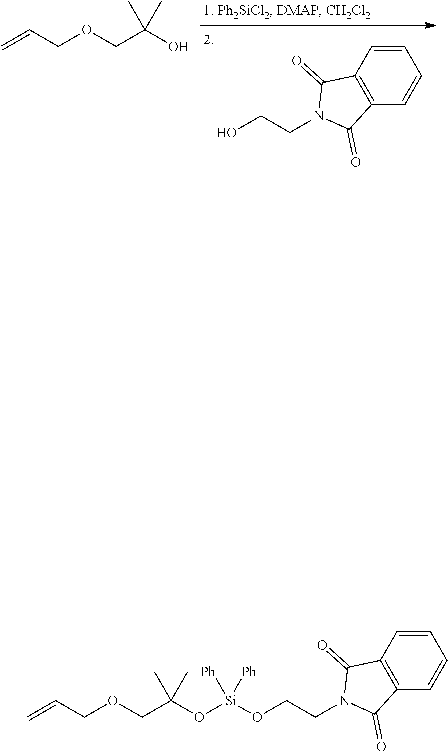

To an oven dried 250 mL round-bottom flask was added DMAP (2.534 g, 2.25 Eq, 20.74 mmol), 1-(allyloxy)-2-methylpropan-2-ol (1.200 g mg, 1 Eq, 9.217 mmol) and capped with septa. The system was purged with argon and dry CH₂Cl₂ (32 mL) was added. The solution was cooled to 0° C. followed by dropwise addition of diphenyldichlorosilane (2.917 g, 2.37 mL, 1.25 Eq, 11.52 mmol). The solution was then allowed to warm to room temperature. After 5 h the first addition was determined complete by TLC. The solution was cooled to 0° C. and N-(2-Hydroxyethyl) phthalimide (1.85 g, 1.05 Eq, 9.68 mmol) was added. The solution was allowed to slowly warm to r.t. and stir overnight. Upon completion the reaction was diluted with sat. sodium bicarbonate and extracted with CH₂Cl₂ (3×20 mL). Combined organic layers were washed with brine and dried over sodium sulfate. Crude product was purified by silica column chromatography (1:9 to 1:3 ethyl acetate:hexanes) to yield the pure product as a clear oil (3.25 g, 70%). ¹H NMR (400 MHz, CDCl₃) δ 7.83-7.76 (m, 2H), 7.73-7.66 (m, 2H), 7.62-7.50 (m, 5H), 7.35-7.28 (m, 2H), 7.25-7.18 (m, 3H), 5.83 (ddt, J=17.3, 10.7, 5.5 Hz, 1H), 5.24-5.09 (m, 2H), 4.02-3.97 (m, 2H), 3.92-3.86 (m, 4H), 3.26 (s, 2H), 1.23 (s, 6H). ¹³C NMR (101 MHz, CDCl₃) δ 168.32, 135.18, 135.00, 134.45, 133.89, 132.33, 129.97, 127.68, 123.28, 116.51, 79.04, 75.58, 72.35, 60.05, 40.06, 27.39. HRMS (ESI-MS) m/z: calculated [M+Na]⁺=524.1870, Found [M+Na]⁺=524.1873

### (9H-Fluoren-9-yl)methyl(6,6-dimethyl-4,4-diphenyl-3,5,8-trioxa-4-silaundec-10-en-1-yl)carbamate

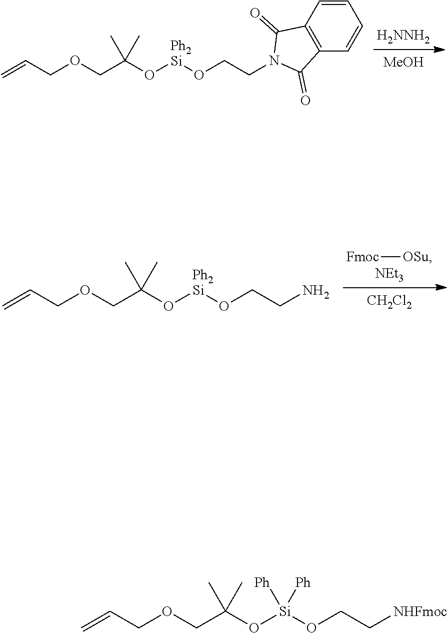

To an oven dried 250 mL round-bottom flask was added 2-(6,6-dimethyl-4,4-diphenyl-3,5,8-trioxa-4-silaundec-10-en-1-yl)isoindoline-1,3-dione (2.64 g, 1 Eq, 5.26 mmol). The vial was capped and purged with nitrogen followed by addition of MeOH (26.3 mL) and then dropwise addition of hydrazine hydrate (1.05 g, 1.02 mL, 4 Eq, 21.1 mmol). Solution let stir at room temperature overnight. Upon completion, reaction mixture diluted with 1M sodium carbonate and 1M oxalic acid and extracted with ethyl acetate (3×30 mL). Then, combined organic extracts washed with brine and dried over sodium sulfate. Volatiles were removed under reduced pressure and material used in the next step without further purification. To an oven dried 250 mL round-bottom flask was added Fmoc-osu (2.13 g, 1.2 Eq, 6.32 mmol), capped, and purged with argon. The crude amine from the first step was dissolved in dry CH₂Cl₂ (26 mL) and added to this flask. Reaction mixture was then cooled to 0° C. and triethylamine (1.28 g, 1.76 mL, 2.4 Eq, 12.6 mmol) added. The solution was allowed to stir at room temperature for 16 hours. Upon completion, the reaction was diluted with water and extracted with CH₂Cl₂ (3×30 mL). Organic layers combined and washed with brine then dried over sodium sulfate. The crude material was purified by silica column chromatography (1:9 to 1:3 ethyl acetate:hexanes) to yield the desired product as a pale yellow oil (1.89 g, 61%). ¹H NMR (400 MHz, CDCl₃) δ 7.80 (d, J=7.6 Hz, 2H), 7.69 (dt, J=6.7, 1.5 Hz, 4H), 7.67-7.63 (m, 2H), 7.47-7.29 (m, 10H), 5.87 (ddt, J=16.3, 10.7, 5.6 Hz, 1H), 5.56 (d, J=5.9 Hz, 1H), 5.29-5.12 (m, 2H), 4.42 (d, J=6.9 Hz, 2H), 4.25 (t, J=6.9 Hz, 1H), 3.94-3.85 (m, 4H), 3.41 (q, J=5.3 Hz, 2H), 3.30 (s, 2H), 1.32 (s, 6H). ¹³C NMR (101 MHz, CDCl₃) δ 156.59, 144.17, 141.41, 135.02, 130.15, 127.81, 127.74, 127.12, 125.17, 120.04, 116.85, 78.87, 75.60, 72.29, 66.67, 62.43, 47.37, 43.27, 27.45. HRMS (ESI-MS) m/z: calculated [M+Na]⁺=616.2495, Found [M+Na]⁺=616.2496.

### General Procedure for Synthesis of β-Hydroxy Isovaleric Esters

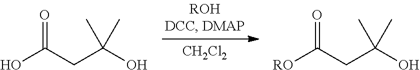

To an oven dried round-bottom flask was added DMAP (0.2 Eq.), CH₂Cl₂ (0.5M), ß-hydroxy Isovaleric Acid (1 Eq.), and alcohol/phenol (1.05-1.2 Eq.). The reaction vessel was lightly purged with argon, cooled to 0° C. and DCC (1.5 Eq.) was added portionwise over 3 minutes. The reaction mixture was then allowed to warm to room temperature and stir until judged complete by TLC (2-16 hours). Crude material was purified by silica column chromatography (1:9 to 1:3 ethyl acetate:hexanes).

### Ethyl 3-hydroxy-3-methylbutanoate

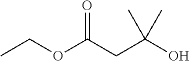

An oven dried 250 mL round-bottom flask was capped with rubber septa and purged with argon. Then anhydrous THF (51 mL) and a 2M solution of LDA in THF (21.2 mL, 0.83 Eq, 42.5 mmol) was added. This mixture was cooled to −78° C. and ethyl acetate (5 mL, 1 Eq, 51.2 mmol) was added dropwise with strong stirring and solution left stirring at −78° C. for 1 hour. After this, acetone (3.79 mL, 1 Eq, 51.2 mmol) was added at −78° C. and the solution left to warm to room temperature and stir for 15 min. The reaction mixture was then diluted with 2M HCl (30 mL) and the organic layer separated. Aqueous layer was then extracted with ethyl acetate (3×30 mL) and combined organic layers were washed with sat. sodium bicarbonate and brine then dried over sodium sulfate. Crude material was purified by vacuum distillation to yield the desired product as a colorless liquid (4.23 g, 56%). All analyses were consistent with previously reported data.^{5 1}H NMR (400 MHz, CDCl₃) δ 4.18 (q, J=7.2 Hz, 2H), 2.48 (s, 2H), 1.33-1.24 (m, 9H).

### Benzyl 3-hydroxy-3-methylbutanoate

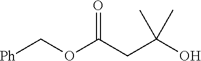

Following the general procedure with benzyl alcohol (0.53 mL, 1.2 Eq, 5.08 mmol) the desired product was obtained as a colorless oil (870 mg, 99%). All analyses were consistent with previously reported data.^{6 1}H NMR (400 MHz, CDCl₃) δ 7.44-7.30 (m, 5H), 5.16 (s, 2H), 3.47 (s, 1H), 2.55 (s, 2H), 1.28 (s, 6H).

### Allyl 3-hydroxy-3-methylbutanoate

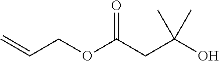

Following the general procedure with allyl alcohol (1.21 mL, 1.05 Eq, 17.8 mmol) the desired product was obtained as a colorless oil (1.77 g, 66%). ¹H NMR (400 MHz, CDCl₃) δ 5.89 (ddt, J=17.1, 10.4, 5.8 Hz, 1H), 5.34-5.19 (m, 2H), 4.59 (dt, J=5.8, 1.4 Hz, 2H), 3.49 (s, 1H), 2.49 (s, 2H), 1.26 (s, 6H). ¹³C NMR (101 MHz, CDCl₃) δ 172.59, 131.88, 118.76, 69.07, 65.30, 46.42, 29.23.

### Methylbut-2-en-1-yl 3-hydroxy-3-methylbutanoate

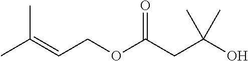

Following the general procedure with prenyl alcohol (1.22 mL, 1.2 Eq, 12 mmol) the desired product was obtained as a colorless oil (1.72 g, 92%). ¹H NMR (400 MHz, CDCl₃) δ 5.27 (ddp, J=8.7, 5.7, 1.4 Hz, 1H), 4.54 (dt, J=7.2, 0.9 Hz, 2H), 3.58 (s, 1H), 2.41 (s, 2H), 1.72-1.62 (m, 6H), 1.20 (s, 6H). ¹³C NMR (101 MHz, CDCl₃) δ 172.81, 139.47, 118.25, 68.95, 61.41, 46.44, 29.11, 25.69, 17.97.

### Tert-butyl 3-hydroxy-3-methylbutanoate

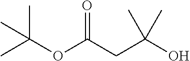

To a flame dried 250 mL round-bottom flask purged with argon was added THF (40 mL) and diisopropylamine (3.66 g, 5.10 mL, 0.97 Eq, 36.2 mmol). The solution was subsequently cooled to 0° C. and n-butyllithium (2.36 g, 14.8 mL, 2.5 molar, 0.99 Eq, 36.9 mmol) was slowly added with strong stirring. This mixture was allowed to react for one hour at 0° C. after which the solution was cooled to −78° C. and tert-butyl acetate (4.33 g, 5.00 mL, 1 Eq, 37.3 mmol) was added dropwise. After another hour of stirring at −78° C., acetone (2.17 g, 2.74 mL, 1 Eq, 37.3 mmol) was added within 30 seconds and the mixture was allowed to stir for a further 10 minutes. The mixture was then warmed to 0° C., diluted with 25 mL water and acidified using 2M HCl and the organic layer separated. The aqueous layer was stripped of its solvents under reduced pressure and extracted with ethyl acetate (3×30 mL). The combined organic extracts were washed with sat. sodium bicarbonate, brine, and dried over sodium sulfate. Material concentrated under reduced pressure to yield the desired product as a colorless oil (5.16 g, 79%). All analyses were consistent with previously reported data.^{5 1}H NMR (400 MHz, CDCl₃) δ 3.80 (s, 1H), 2.39 (s, 2H), 1.47 (s, 9H), 1.26 (s, 6H).

### 2,5-Dioxopyrrolidin-1-yl 3-hydroxy-3-methylbutanoate

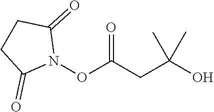

Following the general procedure with N-hydroxysuccinimide (1.22 mL, 1.2 Eq, 12 mmol) and EDC (575 mg, 1 Eq, 3 mmol) the desired product was obtained as a colorless oil (618 mg, 95%). Column chromatography conditions (1:1 to 2:1 ethyl acetate:hexanes). All analyses were consistent with previously reported data. ¹H NMR (400 MHz, CDCl₃) δ 2.85 (br, J=2.8 Hz, 4H), 2.77 (s, J=1.4 Hz, 2H), 1.41-1.37 (s, 6H).

### Methoxybenzyl 3-hydroxy-3-methylbutanoate

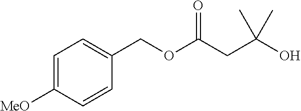 [SMILES: COc1ccc(COC(=O)CC(C)(C)O)cc1]

Following the general procedure with p-methoxybenzyl alcohol (684 mg, 1.2 Eq, 4.95 mmol) the desired product was obtained as a colorless oil (856 mg, 87%). ¹H NMR (400 MHz, CDCl₃) δ 7.24 (d, J=8.7 Hz, 2H), 6.83 (d, J=8.7 Hz, 2H), 5.03 (s, 2H), 3.72 (s, 3H), 3.58 (s, 1H), 2.46 (s, 2H), 1.22 (s, 6H). ¹³C NMR (101 MHz, CDCl₃) δ 172.39, 159.56, 129.99, 127.60, 113.81, 68.89, 65.99, 55.01, 46.47, 29.01.

### 2,3,5,6-Tetrafluorophenyl 3-hydroxy-3-methylbutanoate

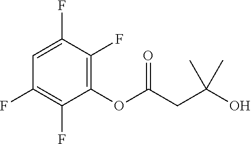 [SMILES: CC(C)(O)CC(=O)Oc1c(F)c(F)cc(F)c1F]

Following the general procedure with 2,3,5,6-tetrafluorophenol (1.69 g, 1.2 Eq, 10.2 mmol) the desired product was obtained as a colorless oil (2.0 g, 89%). ¹H NMR (400 MHz, CDCl₃) δ 7.01 (tt, J=9.9, 7.1 Hz, 1H), 2.87 (s, 2H), 1.41 (s, 6H). ¹³C NMR (101 MHz, CDCl₃) δ 168.25, 103.56 (t), 69.55, 46.25, 29.17. ¹⁹F NMR (376 MHz, CDCl₃) δ −137.70-−139.89 (m), 152.04-153.39 (m).

### 2,6-Difluorophenyl 3-hydroxy-3-methylbutanoate

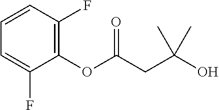 [SMILES: CC(C)(O)CC(=O)Oc1c(F)cccc1F]

Following the general procedure with 2,6-difluorophenol (661 mg, 1.2 Eq, 5.1 mmol) the desired product was obtained as a colorless oil (760 mg, 78%). ¹H NMR (400 MHz, CDCl₃) δ 7.21-7.13 (m, 1H), 7.01-6.93 (m, 2H), 2.99 (s, 1H), 2.84 (s, 2H), 1.40 (s, 6H). ¹³C NMR (101 MHz, CDCl₃) δ 169.05, 156.40, 153.91, 126.65, 112.16, 111.99, 69.28, 46.22, 29.10. ¹⁹F NMR (376 MHz, CDCl₃) δ −125.90.

### 2,4-Difluorophenyl 3-hydroxy-3-methylbutanoate

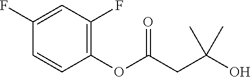 [SMILES: CC(C)(O)CC(=O)Oc1ccc(F)cc1F]

Following the general procedure with 2,4-difluorophenol (661 mg, 1.2 Eq, 5.1 mmol) the desired product was obtained as a colorless oil (950 mg, 97%). ¹H NMR (400 MHz, CDCl₃) δ 7.10 (td, J=8.7, 5.5 Hz, 1H), 6.97-6.84 (m, 2H), 3.08 (br, 1H), 2.79 (s, 2H), 1.39 (s, 6H). ¹³C NMR (101 MHz, CDCl₃) δ 170.08, 160.38 (dd, J=247.7, 10.5 Hz), 154.07 (dd, J=251.9, 12.5 Hz), 134.08 (dd, J=13.1, 4.1 Hz), 124.30 (dd, J=9.9, 2.0 Hz), 111.43 (dd, J=23.1, 3.8 Hz), 105.24 (dd, J=27.0, 22.4 Hz), 69.34, 46.38, 29.25. ¹⁹F NMR (376 MHz, CDCl₃) δ −112.17, −123.09.

### 3-Hydroxy-3-methyl-1-(1H-pyrazol-1-yl)butan-1-one

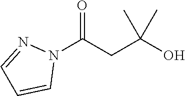

Following the general procedure with 1H-pyrazole (176 mg, 1.2 Eq, 2.59 mmol) the desired product was obtained as a colorless oil (357 mg, 98%). ¹H NMR (400 MHz, CDCl₃) δ 8.29 (dd, J=2.9, 0.7 Hz, 1H), 7.73 (dd, J=1.5, 0.7 Hz, 1H), 6.47 (dd, J=2.9, 1.5 Hz, 1H), 3.34 (s, 2H), 1.37 (s, 6H). ¹³C NMR (101 MHz, CDCl₃) δ 171.26, 144.52, 128.50, 110.16, 69.84, 46.06, 29.60.

### General Procedure for the Synthesis of Dialkoxydiphenylsilanes (DADPS)

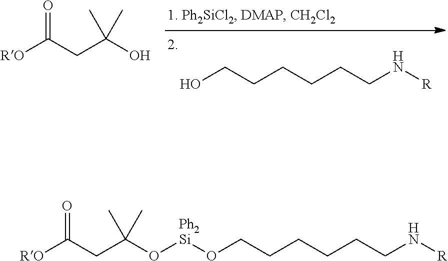

To an oven dried round-bottom flask was added DMAP (2.25 Eq.), ß-hydroxy isovaleric ester (1 Eq.) and capped with septa. The system was purged with argon and dry CH₂Cl₂ (0.2M) was added. The solution was cooled to 0° C. followed by addition of diphenyldichlorosilane (1.25 Eq.). The solution was then allowed to warm to room temperature. Upon completion of the first step as monitored by TLC (5-16 hours) the solution was cooled back to 0° C. The flask was briefly uncapped and the protected amino alcohol (1.05 Eq.) was added in one portion. The flask was recapped, briefly purged with argon, and let warm to room temperature. Upon complete conversion, the reaction was diluted with sat. sodium bicarbonate and extracted with CH₂Cl₂ (3×30 mL). Organic extracts were combined and washed with brine and dried over sodium sulfate. Crude material was purified by silica column chromatography (1:9 to 1:3 ethyl acetate:hexanes).

### Ethyl 14,14-dimethyl-3-oxo-1,12,12-triphenyl-2,11,13-trioxa-4-aza-12-silahexadecan-16-oate

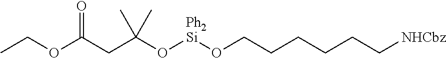

Using general procedure with ethyl 3-hydroxy-3-methylbutanoate (315 mg, 2.15 mmol, 1 Eq.) and benzyl (6-hydroxyhexyl)carbamate (567 mg, 2.26 mmol, 1.05 Eq.) the desired product was obtained as a colorless oil (567 mg, 46%). ¹H NMR (400 MHz, CDCl₃) δ 7.73-7.63 (m, 6H), 7.44-7.30 (m, 11H), 4.16-4.07 (m, 3H), 3.75 (t, J=6.5 Hz, 1H), 3.17 (q, J=6.7 Hz, 1H), 2.65 (s, 2H), 2.59 (s, 1H), 1.63-1.54 (m, 1H), 1.52-1.44 (m, 1H), 1.42 (s, 3H), 1.42 (s, 6H), 1.36-1.25 (m, 1H), 1.25-1.20 (m, 3H). ¹³C NMR (101 MHz, CDCl3) δ 171.99, 136.04, 135.05, 134.61, 130.01, 129.97, 128.59, 127.75, 127.72, 74.37, 74.31, 62.93, 60.69, 60.34, 49.41, 49.00, 32.29, 30.67, 30.20, 25.56, 14.22. HRMS (ESI-MS) m/z: Calculated [M+Na]⁺=600.2758, Found [M+Na]⁺=600.2758.

### Benzyl 14,14-dimethyl-3-oxo-1,12,12-triphenyl-2,11,13-trioxa-4-aza-12-silahexadecan-16-oate

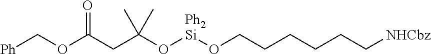

Using general procedure with benzyl 3-hydroxy-3-methylbutanoate (450 mg, 2.15 mmol, 1 Eq.) and benzyl (6-hydroxyhexyl)carbamate (567 mg, 2.26 mmol, 1.05 Eq.) the desired product was obtained as a colorless oil (585 mg, 43%). ¹H NMR (400 MHz, CDCl₃) δ 7.68-7.60 (m, 4H), 7.45-7.27 (m, 16H), 5.12 (br, J=1.9 Hz, 3H), 3.73 (t, J=6.5 Hz, 2H), 3.18 (q, J=6.9 Hz, 2H), 2.66 (s, 2H), 1.63-1.53 (m, 2H), 1.53-1.46 (m, 5H), 1.44 (s, 6H), 1.40-1.33 (m, 1H). ¹³C NMR (101 MHz, CDCl₃) δ 170.76, 136.77, 135.96, 135.02, 134.80, 134.75, 129.98, 128.55, 128.53, 128.35, 128.18, 128.11, 127.70, 74.29, 66.59, 66.27, 62.88, 49.26, 41.10, 32.24, 30.19, 29.95, 26.49, 25.50. HR-MS (ESI-MS) m/z: Calculated [M+Na]⁺=662.2914, Found [M+Na]⁺=662.2950.

### Allyl 14,14-dimethyl-3-oxo-1,12,12-triphenyl-2,11,13-trioxa-4-aza-12-silahexadecan-16-oate

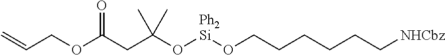

Using general procedure with allyl 3-hydroxy-3-methylbutanoate (200 mg, 1.26 mmol, 1 Eq.) and benzyl (6-hydroxyhexyl)carbamate (334 mg, 1.33 mmol, 1.05 Eq.) the desired product was obtained as a colorless oil (484 mg, 65%). ¹H NMR (400 MHz, CDCl₃) δ 7.67-7.62 (m, 4H), 7.43-7.30 (m, 13H), 5.88 (ddt, J=17.2, 10.4, 5.8 Hz, 1H), 5.33-5.17 (m, 2H), 5.10 (s, 2H), 4.74 (br, 1H), 4.56 (dt, J=5.8, 1.4 Hz, 2H), 3.72 (t, J=6.5 Hz, 2H), 3.17 (q, J=6.7 Hz, 2H), 2.61 (s, 2H), 1.62-1.52 (m, 2H), 1.47 (p, J=7.4 Hz, 2H), 1.41 (s, 6H), 1.39-1.24 (m, 4H). ¹³C NMR (101 MHz, CDCl₃) δ 170.69, 135.08, 134.83, 132.36, 130.04, 128.64, 128.20, 127.75, 118.43, 74.32, 66.70, 65.20, 62.97, 49.31, 41.18, 32.32, 30.23, 30.04, 26.57, 25.59. HRMS (ESI-MS) m/z: Calculated [M+Na]⁺=612.2758, Found [M+Na]⁺=612.2758.

### Methylbut-2-en-1-yl 14,14-dimethyl-3-oxo-1,12,12-triphenyl-2,11,13-trioxa-4-aza-12-silahexadecan-16-oate

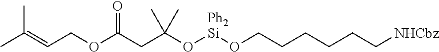

Using general procedure with 3-methylbut-2-en-1-yl 3-hydroxy-3-methylbutanoate (400 mg, 2.15 mmol, 1 Eq.) and benzyl (6-hydroxyhexyl)carbamate (567 mg, 2.26 mmol, 1.05 Eq.) the desired product was obtained as a pale yellow oil (1.08 g, 81%). ¹H NMR (400 MHz, CDCl₃) δ 7.67-7.62 (m, 4H), 7.42-7.30 (m, 11H), 5.30 (tdt, J=5.7, 2.9, 1.4 Hz, 1H), 5.10 (s, 2H), 4.77 (s, 1H), 4.57 (d, J=7.2 Hz, 2H), 3.72 (t, J=6.4 Hz, 2H), 3.17 (q, J=6.9 Hz, 2H), 2.58 (s, 2H), 2.08 (s, 1H), 1.72 (s, 3H), 1.67 (s, 3H), 1.56 (m, 2H), 1.46 (m, 2H), 1.40 (s, 6H), 1.38-1.28 (m, 4H). ¹³C NMR (101 MHz, CDCl₃) δ 171.08, 138.80, 135.08, 134.87, 134.48, 129.99, 128.62, 128.24, 128.19, 127.72, 118.82, 74.34, 66.70, 62.92, 61.33, 49.40, 41.17, 32.31, 30.19, 29.22, 26.56, 25.83, 25.57, 18.08. HRMS (ESI-MS) m/z: Calculated [M+Na]⁺=640.3070, Found [M+Na]⁺=640.3069

### Tert-butyl 14,14-dimethyl-3-oxo-1,12,12-triphenyl-2,11,13-trioxa-4-aza-12-silahexadecan-16-oate

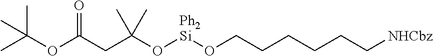

Using general procedure with tert-butyl 3-hydroxy-3-methylbutanoate (400 mg, 2.3 mmol, 1 Eq.) and benzyl (6-hydroxyhexyl)carbamate (606 mg, 2.41 mmol, 1.05 Eq.) the desired product was obtained as a pale yellow oil (741 mg, 53%). ¹H NMR (400 MHz, CDCl₃) δ 7.74-7.68 (m, 4H), 7.45-7.30 (m, 11H), 5.12 (s, 2H), 4.96 (q, J=10.6, 8.2 Hz, 1H), 3.79 (t, J=6.5 Hz, 2H), 3.18 (q, J=6.7 Hz, 2H), 2.54 (s, 2H), 1.69-1.56 (m, 4H), 1.56-1.34 (m, 19H). ¹³C NMR (101 MHz, CDCl₃) δ 170.29, 156.45, 134.99, 134.83, 134.42, 129.89, 128.47, 128.09, 128.02, 127.61, 80.16, 74.37, 66.50, 62.82, 50.59, 47.36, 41.03, 32.21, 30.09, 29.07, 28.10, 26.44, 25.47. HRMS (ESI-MS) m/z: Calculated [M+Na]⁺=628.3070, Found [M+Na]⁺=628.3075

### Methoxybenzyl 14,14-dimethyl-3-oxo-1,12,12-triphenyl-2,11,13-trioxa-4-aza-12-silahexadecan-16-oate

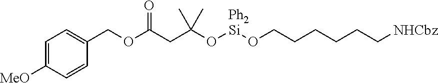

Using general procedure with tert-butyl 3-hydroxy-3-methylbutanoate (411 mg, 1.72 mmol, 1 Eq.) and benzyl (6-hydroxyhexyl)carbamate (477 mg, 1.9 mmol, 1.1 Eq.) the desired product was obtained as a colorless oil (982 mg, 85%). ¹H NMR (400 MHz, CDCl₃) δ 7.65-7.61 (m, 4H), 7.43-7.29 (m, 11H), 7.21 (d, J=8.7 Hz, 2H), 6.82 (d, J=8.7 Hz, 2H), 5.11 (s, 2H), 5.03 (s, 2H), 4.75 (s, 1H), 3.79 (s, 3H), 3.70 (t, J=6.5 Hz, 2H), 3.16 (q, J=6.8 Hz, 2H), 2.61 (s, 2H), 1.59-1.51 (m, 2H), 1.46 (p, J=7.1 Hz, 2H), 1.40 (s, 6H), 1.31 (m, 4H). ¹³C NMR (101 MHz, CDCl₃) δ 170.91, 159.64, 156.48, 136.81, 135.08, 134.86, 134.84, 130.23, 130.01, 128.62, 128.23, 128.18, 127.74, 113.97, 74.36, 66.67, 66.10, 62.93, 55.37, 49.37, 41.16, 32.30, 30.23, 30.02, 26.55, 25.56.

HRMS (ESI-MS) m/z: Calculated [M+Na]⁺=692.3019, Found [M+Na]⁺=692.3018.

### Allyl 3-((((6-(1,3-dioxoisoindolin-2-yl)hexyl)oxy)diphenylsilyl)oxy)-3-methylbutanoate

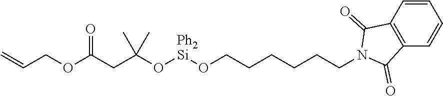

Using general procedure with allyl 3-hydroxy-3-methylbutanoate (100 mg, 0.63 mmol, 1 Eq.) and 2-(6-hydroxyhexyl)isoindoline-1,3-dione (164 mg, 0.66 mmol, 1.05 Eq.) the desired product was obtained as a colorless oil (342 mg, 92%). ¹H NMR (400 MHz, CDCl₃) δ 7.84 (dd, J=5.4, 3.1 Hz, 2H), 7.70 (dd, J=5.5, 3.0 Hz, 2H), 7.65-7.60 (m, 4H), 7.41-7.30 (m, 6H), 5.87 (ddt, J=17.3, 10.4, 5.8 Hz, 1H), 5.32-5.16 (m, 2H), 4.55 (dt, J=5.9, 1.4 Hz, 2H), 3.71 (t, J=6.4 Hz, 2H), 3.66 (t, J=7.3 Hz, 2H), 2.59 (s, 2H), 1.65 (p, J=7.5 Hz, 2H), 1.61-1.52 (m, 3H), 1.39 (s, 6H), 1.42-1.35 (m, 2H), 1.35-1.25 (m, 2H). ¹³C NMR (101 MHz, CDCl₃) δ 170.70, 168.57, 135.09, 134.84, 133.97, 132.33, 130.02, 127.75, 123.29, 118.43, 74.32, 65.20, 63.00, 49.31, 38.15, 32.35, 30.23, 28.75, 26.78, 25.59. HRMS (ESI-MS) m/z: Calculated [M+Na]⁺=608.2444, Found [M+Na]⁺=608.2442

### Allyl 1-(9H-fluoren-9-yl)-14,14-dimethyl-3-oxo-12,12-diphenyl-2,11,13-trioxa-4-aza-12-silahexadecan-16-oate

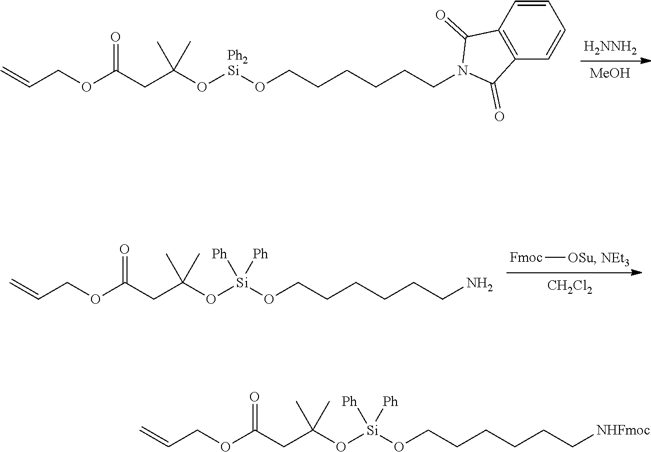

To an oven dried 100 mL round-bottom flask was added allyl 3-((((6-(1,3-dioxoisoindolin-2-yl)hexyl)oxy)diphenylsilyl)oxy)-3-methylbutanoate (1.525 g, 1 Eq, 2.60 mmol). The vial was capped and purged with nitrogen followed by addition of MeOH (26 mL) and then dropwise addition of hydrazine hydrate (521 mg, 0.51 mL, 4 Eq, 10.41 mmol). Solution let stir at room temperature overnight. Upon completion, reaction mixture diluted with 1M sodium carbonate and 1M oxalic acid and extracted with ethyl acetate (3×30 mL). Then, combined organic extracts washed with brine and dried over sodium sulfate. Volatiles were removed under reduced pressure and material used in the next step without further purification. To an oven dried 100 mL round-bottom flask was added Fmoc-osu (1.054 g, 1.2 Eq, 3.12 mmol), capped, and purged with argon. The crude amine from the first step was dissolved in dry CH₂Cl₂ (30 mL) and added to this flask. Reaction mixture was then cooled to 0° C. and triethylamine (632 mg, 0.87 mL, 2.4 Eq, 6.248 mmol) added. Solution let stir at room temperature for 16 hours. Upon completion, the reaction was diluted with water and extracted with CH₂Cl₂ (3×30 mL). Organic layers combined and washed with brine then dried over sodium sulfate. The crude material was purified by silica column chromatography (1:9 to 1:3 ethyl acetate:hexanes) to yield the desired product as a colorless oil (1.22 g, 69%). ¹H NMR (400 MHz, CDCl₃) δ 7.78 (dt, J=7.5, 1.0 Hz, 2H), 7.71-7.65 (m, 4H), 7.62 (dt, J=7.4, 0.9 Hz, 2H), 7.45-7.30 (m, 10H), 5.91 (ddt, J=17.3, 10.4, 5.8 Hz, 1H), 5.36-5.19 (m, 2H), 4.83 (t, J=6.0 Hz, 1H), 4.60 (dt, J=5.8, 1.4 Hz, 2H), 4.44 (d, J=6.9 Hz, 2H), 4.24 (t, J=6.9 Hz, 1H), 3.77 (t, J=6.5 Hz, 2H), 3.18 (q, J=6.7 Hz, 2H), 2.65 (s, 2H), 1.65-1.56 (m, 2H), 1.55-1.47 (m, 2H), 1.45 (s, 6H), 1.43-1.28 (m, 4H). ¹³C NMR (101 MHz, CDCl₃) δ 170.62, 156.48, 144.11, 141.39, 135.04, 134.78, 132.30, 130.01, 127.71, 127.08, 125.09, 120.02, 118.38, 74.27, 66.51, 65.14, 62.94, 49.25, 47.40, 41.10, 32.29, 30.20, 29.99, 26.52, 25.55. HRMS (ESI-MS) m/z: Calculated [M+Na]⁺=700.3070, Found [M+Na]⁺=700.3074.

### 1-(9H-Fluoren-9-yl)-14,14-dimethyl-3,16-dioxo-12,12-diphenyl-2,11,13,17-tetraoxa-21-thia-4-aza-12-silatetracosan-24-oic acid

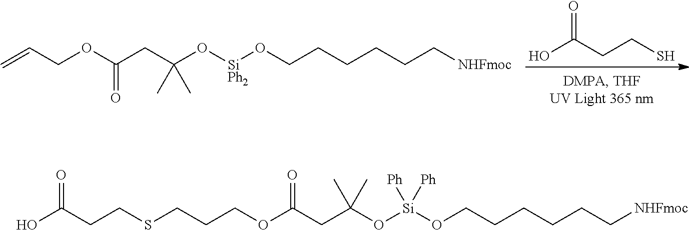

To a 25 mL round-bottom as was added 2,2-dimethoxy-2-phenylacetophenone (19.1 mg, 0.5 Eq, 74.5 μmol), 3-mercaptopropanoic acid (47.4 mg, 39 μL, 3 Eq, 447 μmol), and (9H-fluoren-9-yl)methyl (6,6-dimethyl-8,8-diphenyl-4,7,9-trioxa-8-silapentadec-1-en-15-yl)carbamate (500 mg, 1 Eq, 738 μmol) which were dissolved in dry THF (0.6 mL). The solution was then sparged with nitrogen for 10 minutes to remove oxygen. The reaction mixture was then allowed to stir at room temperature under UV irradiation (365 nm) until judged complete by TLC (4 hours). Upon completion the reaction mixture was diluted with sat. sodium bicarbonate and extracted with ethyl acetate (5×15 mL). The combined organic layers were washed with sat. ammonium chloride and brine then dried over sodium sulfate. The crude material was purified by silica column chromatography (1:1 to 100% ethyl acetate:hexanes) to yield the desired product as a pale yellow oil (69 mg, 59%). ¹H NMR (400 MHz, CDCl₃) δ 7.76 (dt, J=7.6, 1.0 Hz, 2H), 7.66-7.56 (m, 6H), 7.45-7.28 (m, 10H), 4.80 (br, 1H), 4.47-4.39 (m, 2H), 4.21 (m, 1H), 4.14 (m, 2H), 3.73 (t, J=6.3 Hz, 2H), 3.20-3.06 (m, 2H), 2.72 (t, J=7.0 Hz, 2H), 2.57 (ddt, J=21.6, 14.5, 6.7 Hz, 6H), 1.86 (p, J=6.8 Hz, 2H), 1.63-1.53 (m, 2H), 1.51-1.25 (m, 12H). ¹³C NMR (101 MHz, CDCl₃) δ 176.19, 171.01, 156.69, 144.13, 141.46, 135.07, 134.82, 130.10, 127.80, 127.16, 125.15, 120.10, 74.33, 66.69, 62.98, 60.56, 49.37, 47.44, 41.18, 34.55, 32.35, 30.25, 28.74, 26.82, 26.56, 25.59, 21.18, 14.33. HRMS (ESI-MS) m/z: Calculated [M+Na]⁺=806.3159, Found [M+Na]⁺=806.3155

### 1-(9H-Fluoren-9-yl)-10,10-dimethyl-3-oxo-8,8-diphenyl-2,7,9,12-tetraoxa-16-thia-4-aza-8-silanonadecan-19-oic acid

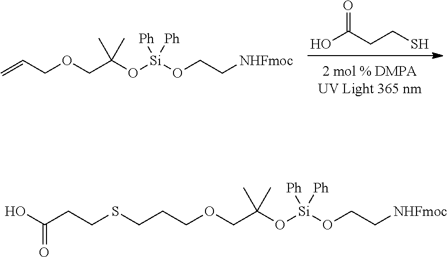

To an oven dried pressure tube was added (9H-fluoren-9-yl)methyl (6,6-dimethyl-4,4-diphenyl-3,5,8-trioxa-4-silaundec-10-en-1-yl)carbamate (494 mg, 1 Eq, 832 μmol), 3-mercaptopropionic acid (88.3 mg, 72.4 μL, 1 Eq, 832 μmol), and DMPA (4.26 mg, 0.02 Eq, 16.6 μmol). The vial was placed under a nitrogen atmosphere through vacuum purge cycles (3 cycles) and then the vial was capped. The vial was then irradiated using UV light (365 nm, 4 W compact lamp) with slow stirring and the whole setup was wrapped in aluminum foil. After 24 hours, full conversion was observed by NMR. The crude mixture was then dissolved in ethyl acetate and washed with sat. sodium bicarbonate (3×5 mL), sat. ammonium chloride (1×5 mL), and brine. The organic layer was then dried over sodium sulfate and concentrated under reduced pressure to yield the desired product as a pale-yellow wax (541 mg, 93%). ¹H NMR (400 MHz, CDCl₃) δ 7.78 (d, J=7.6 Hz, 2H), 7.64 (dd, J=19.6, 4.5 Hz, 6H), 7.37 (ddt, J=25.2, 14.3, 5.2 Hz, 10H), 4.41 (d, J=6.9 Hz, 2H), 4.23 (t, J=6.8 Hz, 1H), 3.90-3.69 (m, 2H), 3.46-3.16 (m, 6H), 2.73 (t, J=7.2 Hz, 2H), 2.57 (dt, J=22.3, 7.3 Hz, 4H), 1.77 (t, J=6.9 Hz, 1H), 1.27 (m, 8H). ¹³C NMR (101 MHz, CDCl₃) δ 156.67, 144.05, 141.36, 134.95, 134.46, 130.13, 127.78, 127.72, 127.09, 125.09, 120.00, 79.54, 75.56, 69.71, 66.70, 62.32, 47.28, 43.21, 34.66, 29.55, 28.83, 27.35, 26.80. HRMS (ESI-MS) m/z: Calculated [M+Na]⁺=722.2584, Found [M+Na]⁺=722.2601

### 3-((3-((3-hydroxy-3-methylbutanoyl)oxy)propyl)thio)propanoic acid

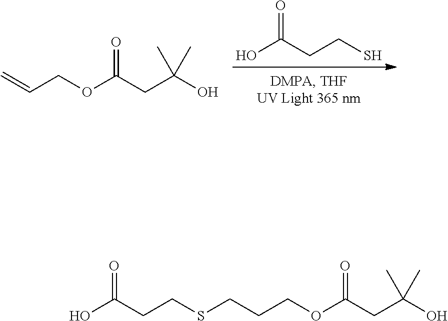 [SMILES: C=CCOC(=O)CC(C)(C)O ; O=C(O)CCS ; CC(C)(O)CC(=O)OCCCSCCC(=O)O]

To a one-dram scintillation vial equipped with stir bar was added allyl 3-hydroxy-3-methylbutanoate (100 mg, 0.63 mmol, 1 Eq.), 3-mercaptopropionic acid (201 mg, 165 μL, 1.90 mmol, 3 Eq.), DMPA (81 mg, 0.32 mmol, 0.5 Eq.), and THF (2.4 mL). The solution was sparged with argon for 10 minutes and then subjected to UV irradiation (365 nm, 6 W handheld lamp) for 4 h. The crude material was purified by silica column chromatography (1:1 ethyl acetate:hexanes) to yield the desired product as a white solid (102 mg, 61%). ¹H NMR (400 MHz, CDCl₃) δ 6.25 (br, 1H), 4.20 (t, J=6.3 Hz, 2H), 2.77 (t, J=7.2 Hz, 2H), 2.62 (dt, J=11.6, 7.1 Hz, 4H), 2.50 (s, 2H), 1.92 (p, J=6.7 Hz, 2H), 1.28 (s, 6H). ¹³C NMR (101 MHz, CDCl₃) δ 176.82, 172.94, 69.49, 63.26, 46.42, 34.73, 29.20, 28.64, 28.53, 26.83. HRMS (ESI-MS) m/z: Calculated [M+Na]⁺=287.0929, Found [M+Na]⁺=287.0952.

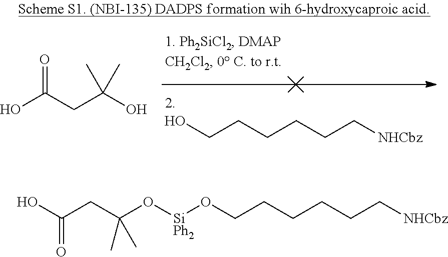

Scheme S2. Initial screen of DADPS formation conditions.

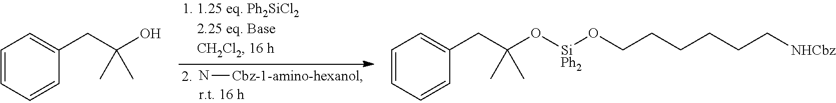

| Entry # | Base | Temperature (1st Step) | Yield |
|---|---|---|---|
| 1 | DMAP | 0° C. to 25° C. | 59% |
| 2 | NEt₃ | 0° C. to 25° C. | 18% |
| 3 | NEt₃ | 0° C. to 40° C. | 22% |

Scheme S3. Preparation of DADPS ester analogues.

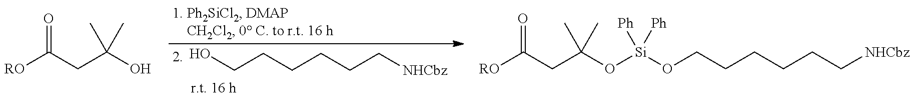

| Entry | R | Yield |
|---|---|---|
| 1 | Et | 45.5% |
| 2 | Bn | 43% |
| 3 | Allyl | 64.9% |
| 4 | Prenyl | 81.4% |
| 5 | t-Bu | 53.3% |
| 6 | PMB | 85% |

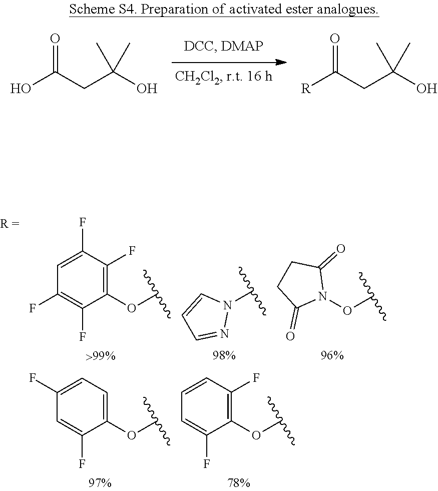

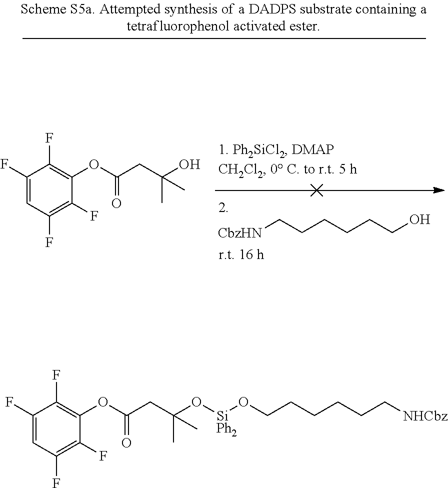

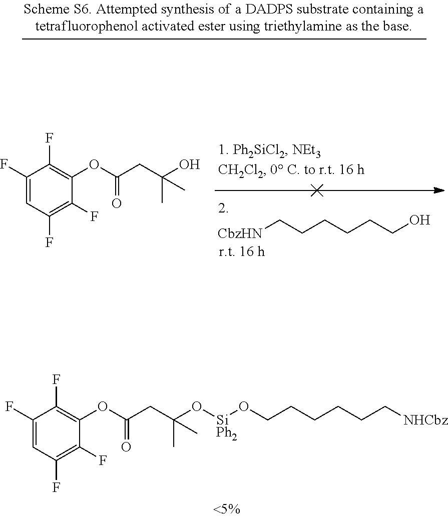

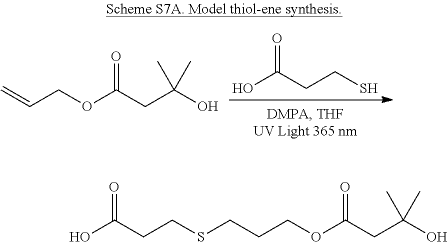 [SMILES: C=CCOC(=O)CC(C)(C)O ; O=C(O)CCS ; CC(C)(O)CC(=O)OCCCSCCC(=O)O]

Scheme S7B. Deprotection condition for DADPS reagent bearing an allyl ester and Cbz-
protected amine.

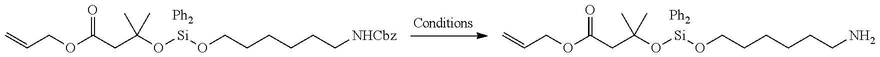

| Entry | Conditions | Time | Result |
|---|---|---|---|
| 1 | Pd(OAc)₂, Et₃SiH, NEt₃ | 2 h | Decomposition |
| | CH₂Cl₂ | | |
| 2 | TMSI, CDCl₃ | 6 min | Decomposition |
| 3 | KOH, MeOH | 2 h | Decomposition |
| 4 | PdCl₂, Et₃SiH, NEt₃ | 2 h | Observed hydrogenation of alkene |
| | CH₂Cl₂ | | and Cbz deprotection |
| 5 | H₂, Pd/C, MeOH | 30 min | Observed hydrogenation of alkene |
| | | | and Cbz deprotection |
| 6 | H₂, Pd/C, MeOH | 15 min | Observed hydrogenation of alkene |
| | | | and Cbz deprotection |
| 7 | H₂, Pd/C, MeOH | 5 min | Observed hydrogenation of alkene |
| | | | and Cbz deprotection |
| 8 | H₂, Pd/C, MeOH | 1 min | Observed hydrogenation of alkene |
| | | | and Cbz deprotection |

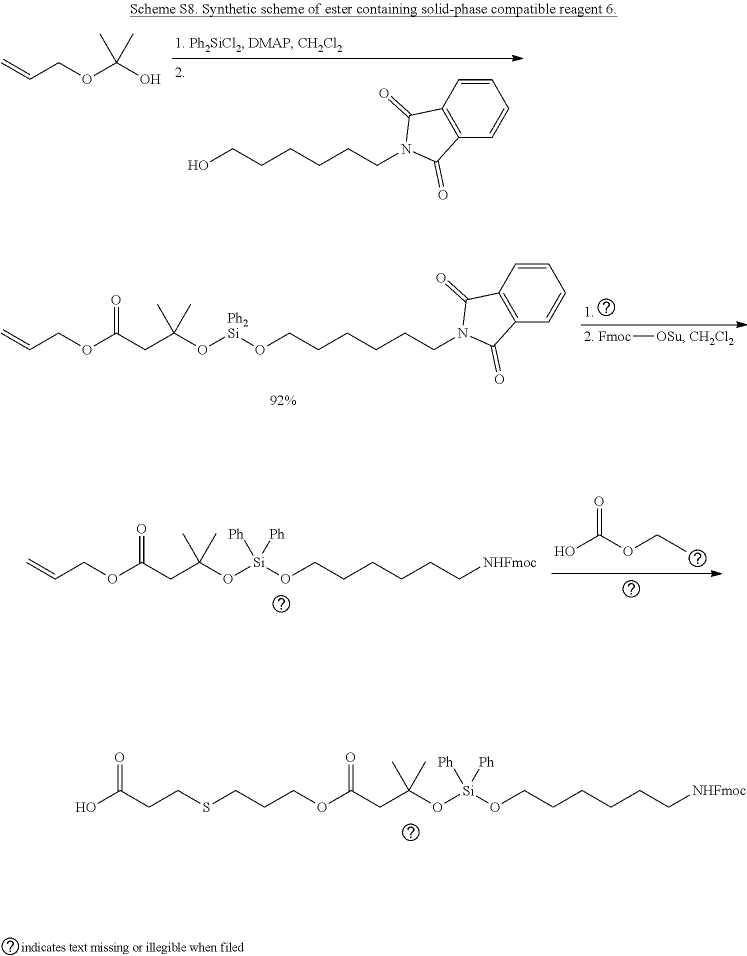

TABLE S1
Ester Cleavage Screen

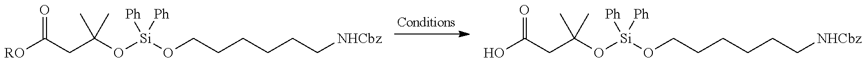

| Entry | R | Conditions | Result |
|---|---|---|---|
| 1 | Et | NEt₃, LiBr, MeCN | No reaction |
| 2 | Et | 1M NaOH, 1:1 MeOH:H₂O | No observable desired product |
| 3 | Et | 1M LiOH, 1:1 THF:H₂O | No observable desired product |
| 4 | Et | 5M NaOH in MeOH | Recovered sm and methyl ester |
| | | r.t. to 50° C. | |
| 5 | Bn | H₂, Pd/C, MeOH | Cbz deprotection |
| 6 | Bn | H₂, Pd/C, HFIP | Cbz deprotection |
| 7 | Allyl | Pd(PPh₃)₄, PhSiH₃ | Decomposition |
| | | CH₂Cl₂ | |
| 8 | Allyl | Pd(PPh₃)₄, 4-MethylMorpholine | Decomposition |
| | | THF | |
| 9 | PMB | DDQ | Decomposition |
| 10 | Bn | SnCl₄, CH₂Cl₂ | Cleavage of ether |
| 11 | Prenyl | CeCl₃, Nal | Cleavage of ether |
| | | 80° C., 2 h | |
| 12 | Prenyl | BF₃•OEt₂, Dioxane | Cleavage of ether |
| 13 | Et | TMSI, I₂, NEt₃ | Cleavage of ether |

### Biology Methods

### Cell Culture and Preparation of Cell Lysates.

Cell culture reagents including Dulbecco's phosphate-buffered saline (DPBS), Dulbecco's modified Eagle's medium (DMEM)/high glucose media, Roswell Park Memorial Institute (RPMI) media, trypsin-EDTA and penicillin/streptomycin (Pen/Strep) were purchased from Fisher Scientific. Fetal Bovine Serum (FBS) were purchased from Avantor Seradigm (lot #214B17). All cell lines were obtained from ATCC and were maintained at a low passage number (<20 passages). HEK293T (ATCC: CRL-3216) cells were cultured in DMEM supplemented with 10% FBS and 1% antibiotics (Penn/Strep, 100 U/mL). H661 (ATCC: HTB-183), HCT-15 (ATCC: CCL-225), Jurkat (ATCC: TIB-152), MOLT-4 (ATCC: CRL-1582) and H2122 (ATCC: CRL5985) cells were cultured in RPMI-1640 supplemented with 10% FBS and 1% antibiotics (Penn/Strep, 100 U/mL). HEC-1-B (ATCC: HTB-113) cells were cultured in EMEM supplemented with 10% FBS and 1% antibiotics (Penn/Strep, 100 U/mL). Media was filtered (0.22 μm) prior to use. Cells were maintained in a humidified incubator at 37° C. with 5% CO2. Cell lines were tested for Mycoplasma using the Mycoplasma Detection Kit (InvivoGen). Cells were harvested by centrifugation (4,500 g, 5 min, 4° C.), washed twice with cold DPBS, resuspended in DPBS, sonicated, and clarified by centrifuging (21,000 g, 10 min, 4° C.). The lysates were then transferred to a new microcentrifuge tube. Protein concentrations were determined using a Bio-Rad DC protein assay kit from Bio-Rad Life Science (Hercules, CA) and the lysate diluted to the working concentrations indicated below.

### Proteomic Sample Preparation.

HEK293T proteome (100 μL of 2 mg/mL) was first labeled with IAA 1 or other reagents (2 μL of 100 mM stock solution in DMSO, final concentration=2 mM) for 1 h at ambient temperature. CuAAC was performed with biotin-azide 2 or other reagents (2 μL of 200 mM stock in DMSO, final concentration=4 mM), TCEP (2 μL of fresh 50 mM stock in water, final concentration=1 mM), TBTA (6 μL of 1.7 mM stock in DMSO/t-butanol 1:4, final concentration=100 μM), CuSO4 (2 μL of 50 mM stock in water, final concentration=1 mM), and 0.2% SDS for 1 h at ambient temperature. After CuAAC labeling, each sample was treated with 0.5 μL benzonase (Fisher Scientific, 70-664-3) for 30 min at 37° C. For each 100 μL sample (1 mg/mL protein concentration), 20 μL Sera-Mag SpeedBeads Carboxyl Magnetic Beads, hydrophobic (GE Healthcare, 65152105050250) and 20 μL Sera-Mag SpeedBeads Carboxyl Magnetic Beads, hydrophilic (GE Healthcare, 45152105050250) were mixed and washed with water for three times. The bead slurries were then transferred to the CuAAC samples, incubated for 5 min at RT with shaking (1000 rpm). Absolute ethanol (400 μL) was added to each sample, and the samples were incubated for 5 min at RT with shaking (1000 rpm). Samples were then placed on a magnetic rack, washed three times with 80% ethanol in water (400 μL). After washing, beads were resuspended in 200 μL 2 M urea in 0.5% SDS/PBS. DTT (10 μL of 200 mM stock in water, final concentration=10 mM) was added into each sample and the sample was incubated at 65° C. for 15 min. Then, iodoacetamide (10 μL of 400 mM stock in water, final concentration=20 mM) was added and the solution was incubated for 30 min at 37° C. with shaking in the dark. Absolute ethanol (400 μL) was added to each sample, and the samples were incubated for a further 5 min at RT with shaking (1000 rpm). Beads were washed three times with 80% ethanol in water (400 μL). Next, beads were resuspended in 200 μL 2 M urea in PBS and 2 μL trypsin solution (Worthington Biochemical, LS003740, 1 mg/mL in 666 μL of 50 mM acetic acid and 334 μL of 100 mM CaCl2)) was added. Digest was overnight at 37° C. with shaking. After digestion, ˜4 mL acetonitrile (>95% of the final volume) was added to each sample and the mixtures were incubated for 10 min at RT with shaking (1000 rpm). The beads were then washed three times with 1 mL acetonitrile each with a magnetic rack. Peptides were eluted from SP3 beads with 100 μL of 2% DMSO in Molecular Biology Grade (MB) water for 30 min at 37° C. with shaking (1000 rpm). The elution was repeated again with 100 μL of 2% DMSO in MB water. Two eluents were combined.

### Avidin Enrichment of Labeled Peptides.

For each sample clicked with biotin azide, 50 μL of NeutrAvidin Agarose resin slurry (Pierce, 29200) was washed one time in 10 mL IAP buffer (50 mM MOPS pH 7.2, 10 mM sodium phosphate, and 50 mM NaCl buffer) and then resuspended in 500 μL IAP buffer. Peptide solutions eluted from SP3 beads were then transferred to the NeutrAvidin Agarose resin suspension, and the samples were rotated for 2 h at RT. For each sample clicked with a DADPS cleavable azide, 50 L of Streptavidin Agarose resin slurry (Pierce, 20353) was washed one time in 10 mL PBS and then resuspended in 500 μL PBS. Peptide solutions eluted from SP3 beads were then transferred to the Streptavidin Agarose resin suspension, and the samples were rotated for 2 h at RT.

After incubation, the beads were pelleted by centrifugation (21,000 g, 1 min) and washed twice with 1 mL PBS each and then twice with 1 mL water each. NeutrAvidin-bound peptides were eluted with 60 μL of 80% acetonitrile in MB water with 0.1% FA for 10 min at RT. The elution was repeated for 10 min at 72° C. The elution was repeated once more for 10 min at RT. Streptavidin-bound peptides were eluted with 200 μL of 2% formic acid in MB water for 30 min at RT. The elution was repeated once more with 80% acetonitrile in MB water for 2 min at RT. The combined eluants were dried (SpeedVac), then reconstituted with 5% acetonitrile and 1% FA in MB water and analyzed by LC-MS/MS.

### Liquid-Chromatography Tandem Mass-Spectrometry (LC-MS/MS) Analysis.

The samples were analyzed by liquid chromatography tandem mass spectrometry using a Thermo Scientific™ Orbitrap Eclipse™ Tribrid™ mass spectrometer or coupled with a High Field Asymmetric Waveform Ion Mobility Spectrometry (FAIMS) Interface. Peptides were fractionated S21 online using a 18 cm long, 100 μM inner diameter (ID) fused silica capillary packed in-house with bulk C18 reversed phase resin (particle size, 1.9 μm; pore size, 100 Å; Dr. Maisch GmbH). The 70-minute water-acetonitrile gradient was delivered using aThermo Scientific™ EASY-nLC™ 1200 system at different flow rates (Buffer A: water with 3% DMSO and 0.1% formic acid and Buffer B: 80% acetonitrile with 3% DMSO and 0.1% formic acid). The detailed gradient includes 0-5 min from 3% to 10% at 300 nL/min, 5-64 min from 10% to 50% at 220 nL/min, and 64-70 min from 50% to 95% at 250 nL/min buffer B in buffer A. Data was collected with charge exclusion (1, 8, >8). Data was acquired using a Data-Dependent Acquisition (DDA) method consisting of a full MS1 scan (Resolution=120,000) followed by sequential MS2 scans (Resolution=15,000) to utilize the remainder of the 1 second cycle time. Precursor isolation window and normalized collision energy were set as described in the study. Table S7. Conditions of Liquid-chromatography (LC) Parameter Condition Column 100 μM ID fused silica capillary packed in-house with bulk C18 reversed phase resin (particle size, 1.9 μm; pore size, 100 Å; Dr. Maisch GmbH) Mobile phase Buffer A: water with 3% DMSO and 0.1% formic acid Buffer B: 80% acetonitrile with 3% DMSO and 0.1% formic acid Gradient and flow rate 0-5 min, 3-10% B, 300 nL/min 5-64 min, 10-50% B, 220 nL/min 64-70 min, 40-95% B, 250 nL/min Run time 70 minutes Injection volume 5 uL.

### Protein and Peptide Identification.

Raw data collected by LC-MS/MS were searched with MSFragger (v3.4 and v3.5) and FragPipe (v17.1 and 18.0). For closed search, the proteomic workflow and its collection of tools was set as default. Precursor and fragment mass tolerance was set as 20 ppm. Missed cleavages were allowed up to 1. Peptide length was set 7-50 and peptide mass range was set 500-5000. Cysteine residues were searched with differential modifications as described in the study. For labile search, mass offsets were set restricted to cysteines. Y ion masses and diagnostic fragment masses were set for different proteomic samples. PTM-Shepherd was enabled for localization. A sample workflow can be found attached. Calibrated and deisotoped spectrum files produced by FragPipe were retained and reused for this analysis.

### Data Analysis and Processing.

After MS search with MSFragger, raw files and identification files were imported to PDV for MS spectra annotation. Frequency distribution and intensity of the fragment ions and peptide remainder ions were calculated based on the output of PTMShepherd as mean of all replicates (See supplementary data tables). Mean of the number of PSMs and peptides of all replicates were reported as bar plots. Additionally, venn diagrams were constructed to display the number of common cysteine peptides identified shared between various experiments.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A compound having a structure represented by formula I or a salt thereof: 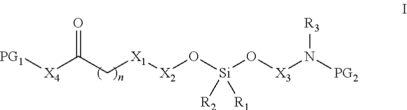
wherein
X₁ and X₄ are each independently O, S, or NR₄;
X₂ and X₃ are each independently alkylene;
PG₁ is H, an oxygen protecting group, or a sequence of amino acids;
PG₂ is H, a nitrogen protecting group or a sequence of amino acids; and
R₁ and R₂ are each independently alkyl, aralkyl, or aryl;
R₃ and R₄ are each independently H or alkyl; and
n is 1, 2, 3, 4, 5, 6, 7, 8, 9, or 10.

2. The compound of claim 1, wherein X₁ is S.

3. The compound of claim 1 or 2, wherein X₂ is alkyloxyalkyl.

4. The compound of any one of claims 1-3, wherein X₂ is substituted with alkyl, alkenyl, alkynyl, ester, amido, aryl, or heteroaryl.

5. The compound of any one of claims 1-3, wherein X₂ is substituted with alkyl (e.g., methyl).

6. The compound of any one of claims 1-5, wherein X₃ is methyl, ethyl, propyl, butyl, pentyl, hexyl, heptyl, or octyl.

7. The compound of claim 6, wherein X₃ is hexyl.

8. The compound of claim 6, wherein X₃ is propyl.

9. The compound of any one of claims 1-8, wherein X₃ is substituted with alkyl, alkenyl, alkynyl, ester, amido, aryl, or heteroaryl.

10. The compound of any one of claims 1-9, wherein R₃ is H.

11. The compound of any one of claims 1-10, wherein n is 2.

12. The compound of any one of claims 1-11, wherein X₄ is O.

13. The compound of any one of claims 1-11, wherein X₄ is NR₄.

14. The compound of claim 13, wherein R₄ is H.

15. The compound of any one of claims 1-14, wherein PG₁ is alkyl, benzyl, or heteroaryl.

16. The compound of any one of claims 1-14, wherein PG₁ is H.

17. The compound of any one of claims 1-14, wherein PG₁ is a sequence of amino acids (e.g., 1-10 amino acids).

18. The compound of any one of claims 1-17, wherein the compound has a structure represented by formula Ia or a salt thereof: 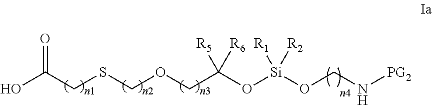
wherein
n1, n2, n3, and n4 are each independently 1, 2, 3, 4, 5, 6, 7, 8, 9, or 10;
R₅ and R₆ are each independently alkyl, alkenyl, alkynyl, ester, amido, aryl, or heteroaryl.

19. The compound of claim 18, wherein n1 is 2.

20. The compound of claim 18 or 19, wherein n2 is 3.

21. The compound of any one of claims 18-20, wherein n3 is 1.

22. The compound of any one of claims 18-21, wherein n4 is 2.

23. The compound of any one of claims 18-21, wherein n4 is 6.

24. The compound of any one of claims 18-23, wherein R₅ is alkyl (e.g., methyl).

25. The compound of any one of claims 18-24, wherein R₆ is alkyl (e.g., methyl).

26. The compound of any one of claims 1-25, wherein R₁ is aryl (e.g., phenyl).

27. The compound of any one of claims 1-26, wherein R₂ is aryl (e.g., phenyl).

28. The compound of any one of claims 1-27, wherein PG₂ is alkyl, arakyl, carbamyl, heteroaryl, hetercyclyl, acetyl, or sulfonyl.

29. The compound of any one of claims 1-27, wherein PG₂ is Fmoc.

30. The compound of any one of claims 1-27, wherein PG₂ is sequence of amino acids (e.g., 1-10 amino acids).

31. The compound of claim 1, wherein the compound is selected from: 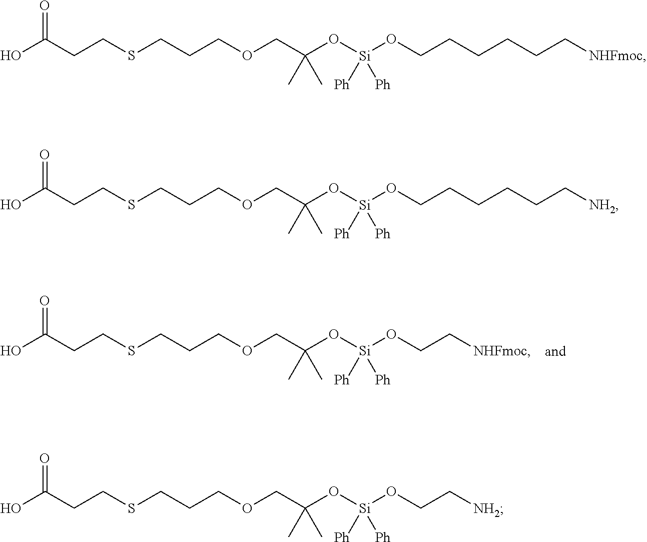
a salt thereof.

32. A method of synthesizing a chemoproteomic capture reagent comprising:
contacting a solid support with an enrichment handle, thereby creating a solid support-enrichment handle conjugate;
contacting the solid support-enrichment handle conjugate with a solid-phase compatible cleavable linker, thereby creating a solid support-enrichment handle-solid-phase compatible cleavable linker conjugate;
contacting the solid support-enrichment handle-solid-phase compatible cleavable linker conjugate with a click capture amino acid, thereby creating a solid support-enrichment handle-solid-phase compatible cleavable linker-click capture amino acid conjugate; and
contacting the solid support-enrichment handle-solid-phase compatible cleavable linker-click capture amino acid conjugate with an isotopically labelled amino acid, thereby creating a solid support-enrichment handle-solid-phase compatible cleavable linker-click capture amino acid-isotopically labelled amino acid conjugate.

33. The method of claim 32, further comprising cleaving the support-enrichment handle-solid-phase compatible cleavable linker-isotopically labelled amino acid conjugate from the solid support.

34. The method of claim 32 or 33, wherein the solid support is a resin.

35. The method of any one of claims 32-34, wherein the resin is a chlorotrityl resin.

36. The method of any one of claims 32-35, wherein the enrichment handle is an amino acid substituted with biotin (e.g., an amino acid having a side chain substituted with biotin).

37. The method of claim 36, wherein the amino acid is a naturally occurring amino acid (e.g., lysine or cysteine).

38. The method of any one of claims 32-37, wherein the solid-phase compatible cleavable linker is the compound of any one of claims 1-31.

39. The method of any one of claims 32-38, wherein the click capture amino acid is an azide containing amino acid (e.g., an amino acid having a side chain substituted with an azide).

40. The method of any one of claims 32-39, wherein the isotopically labelled amino acid is a naturally occurring amino acid (e.g., valine or alanine).

41. The method of claim 40, wherein the isotopically labelled amino acid is enriched with C¹³ or N¹⁵.

42. The method of any one of claims 32-41, wherein the support-enrichment handle-solid-phase compatible cleavable linker-isotopically labelled amino acid conjugate is cleaved from the solid support using acid (e.g., hydrochloric acid).

43. A method of identifying a binding site comprising:
contacting substrate with an alkyne, thereby creating a substrate-alkyne conjugate;
contacting the substrate-alkyne conjugate with the chemoproteomic capture reagent of any one of claims 32-42, thereby creating a chemoproteomic capture reagent-substrate conjugate;
digesting the chemoproteomic capture reagent-substrate conjugate, thereby creating a digested substrate-chemoproteomic capture reagent conjugate;
contacting the digested substrate-chemoproteomic capture reagent conjugate with an enrichment agent;
cleaving the digested substrate-chemoproteomic capture reagent conjugate, thereby creating a digested substrate-isotopically labelled amino acid conjugate; and
determining the molecular weight of the digested substrate-isotopically labelled amino acid conjugate, thereby identifying the binding site.

44. The method of claim 43, wherein the substrate is a protein.

45. The method of claim 44, wherein the protein is formed from cell lysation.

46. The method of any one of claims 43-45, wherein contacting the substrate-alkyne conjugate with the chemoproteomic capture reagent of any one of claims 32-42, forming a triazole linking the chemoproteomic capture reagent to the substrate.

47. The method of any one of claims 43-46, wherein digesting the chemoproteomic capture reagent-substrate conjugate comprising contacting the chemoproteomic capture reagent-substrate conjugate with a digestion enzyme (e.g., trypsin).

48. The method of any one of claims 43-47, wherein the enrichment agent is a protein that binds biotin (e.g., avidin or streptavidin).

49. The method of any one of claims 43-48, wherein cleaving the digested substrate-chemoproteomic capture reagent conjugate comprising contacting the digested substrate-chemoproteomic capture reagent conjugate with acid (e.g., formic acid).
